(19) United States
(12) Patent Application Publication
(10) Pub. No.: US 2008/0317885 A1
(43) Pub. Date: Dec. 25, 2008

(54) Compositions and Methods for Treating and Preventing Inflammatory and/or Degenerative Processes in Humans and Other Animals

(76) Inventor: Donald J. Baker, Greenvale, NY (US)

Correspondence Address: Peter Rogalskyj;Rogalskyj & Weyand, P.O. Box 44, Livonia, NY, 14487-0044, US

(21) Appl. No.: 11/884,170

(22) PCT Filed: Jul. 15, 2006
(86) PCT No.: PCT/US06/27265

§ 371 Date: Feb. 25, 2008

Related U.S. Application Data
(60) Provisional application No. 60/699,982, filed on Jul. 15, 2005.

(51) Int. Cl. A61K 31/352; A61P 29/00; A61K 31/353; A61K 31/19; A61P 25/00; A61K 36/53; A61K 36/484; A61K 36/54; A61K 36/57; A61K 36/37

(52) U.S. Cl. 424/739; 514/456; 514/557; 424/725; 424/757

(57) ABSTRACT

Disclosed are compositions useful for treating Alzheimer's disease, atherosclerosis, arteriosclerosis, osteoarthritis and other degenerative joint diseases, Huntington's chorea, Parkinson's disease, optic atrophy, retinitis pigmentosa, macular degeneration, muscular dystrophy, aging-associated degenerative processes, asthma, dermatitis, laminitis, pemphigoid, pemphigus, reactive airway disease (e.g., COPD, IAD), inflammatory bowel disease (e.g., Crohn's disease, ulcerative colitis), multiple sclerosis, rheumatoid arthritis, periodontal disease, systemic lupus erythematosus, sarcoidosis, psoriasis, type I diabetes, ischemia-reperfusion injury, chronic inflammatory diseases, geriatric wasting, cancer cachexia, cachexia associated with chronic inflammation, sick feeling syndrome, and other inflammatory and/or degenerative diseases, disorders, conditions, and processes in humans and other animals. In one embodiment, the compositions include at least 4 of the following: a MMP1 inhibitor, a MMP2 inhibitor, a MMP3 inhibitor, a MMP7 inhibitor, a MMP9 inhibitor, an ADAMTS-4 inhibitor, a MMP13 inhibitor, and a MMP14 inhibitor. In another embodiment, the compositions include a curcuminoid, a polymethoxylated flavone, a catechin, and a boswellic acid.

The present application claims the benefit of U.S. Provisional Patent Application Ser. No. 60/699,982 filed Jul. 15, 2005, which provisional patent application is hereby incorporated by reference.

### FIELD OF THE INVENTION

The subject invention is directed to methods and compositions for treating and preventing inflammatory and/or degenerative processes in animals and humans.

### BACKGROUND OF THE INVENTION

Humans and many animals are afflicted with a variety of inflammatory and/or degenerative diseases, disorders, conditions, and other processes. Such inflammatory and/or degenerative processes include Alzheimer's Disease, asthma, atherosclerosis, dermatitis (e.g., atopic dermatitis, auto-immune dermatitis, allergic chronic contact dermatitis, and environmental chronic contact dermatitis), laminitis (e.g., chronic laminitis), Bullous pemphigoid, reactive airway diseases and processes (e.g., chronic obstructive pulmonary disease (“COPD”), inflammatory airway disease (“IAD”), etc.), gout, inflammatory bowel disease, ischemia-reperfusion injury, multiple sclerosis, osteoarthritis, periodontal disease, psoriasis, rheumatoid arthritis, sarcoidosis, systemic lupus erythematosus, type I diabetes, and ulcerative colitis.

For example, osteoarthritis (osteoarthrosis) is a degenerative process that is a major cause of invalidism in both humans and other animals. Osteoarthritis is the most common form of all articular disorders. In humans, it first appears asymptomatically in the second or third decades of life and becomes almost universal by age 70. Almost all persons by the age of 40 have some pathological changes in weight bearing joints, although relatively few people are symptomatic.

The etiology of osteoarthritis is unknown. It appears to be the result of a complex system of interacting mechanical, biological, biochemical, enzymatic, and immunologic mechanisms. When homeostatic control systems are overwhelmed, the clinical events follow. Many mechanisms can initiate the cellular and tissue events that constitute the disease condition. Such mechanisms include: congenital joint abnormalities; genetic defects; infectious, metabolic, endocrine, and neuropathic diseases; virtually any disease process that alters the normal structure and function of hyaline cartilage; and acute or chronic trauma to the hyaline cartilage or tissue surrounding same.

Analgesics, anti-inflammatory agents, (both steroidal and non-steroidal), and immunosuppressive agents are used to attempt to manage this and other degenerative disorders. However, these agents are not curative; they only function to relieve the pain and other symptoms associated with the disorder, and perhaps slow the progression by subduing the inflammatory response.

Chronic inflammatory conditions, such as inflammatory airway disease complex and other reactive airway diseases, auto-immune dermatitis, chronic contact dermatitis, inflammatory bowel disease, and chronic laminitis, are currently treated with NSAIDS and other anti-cytokine strategies, glucocorticoids, and immunosuppressive agents, all of which are fraught with negative side effects.

There is a need for improved compositions and methods for treating degenerative joint disease, such as osteoarthritis, and other inflammatory and/or degenerative processes, and the present invention is directed to meeting this need.

### SUMMARY OF THE INVENTION

The present invention relates to a composition for treating an inflammatory and/or degenerative process in a human or other animal. The composition includes four or more of the following: a MMP 1 inhibitor, a MMP 2 inhibitor, a MMP 3 inhibitor, a MMP 7 inhibitor, a MMP 9 inhibitor, an ADAMTS-4 inhibitor, a MMP 13 inhibitor, and a MMP 14 inhibitor.

The present invention also relates to a composition for treating an inflammatory and/or degenerative process in a human or other animal in which the composition includes a curcuminoid, a polymethoxylated flavone, a catechin, and a boswellic acid.

### BRIEF DESCRIPTION OF THE DRAWINGS

FIG. 1 is a schematic diagram of a proposed unregulated cycle of amplified cytokine/chemokine/MMP production that is believed to explain why an excess of MMPs can lead to chronic, progressive degradation of tissue (by MMPS) and chronic inflammation of tissue (by chemokines and cytokines).

FIGS. 2A-2B are schematic illustrations of the MMP/TIMP/cytokine axis. FIG. 2A shows a balanced axis where TIMPs regulate levels and activity of MMPs, ADAMs, and ADAMTSs, which have downstream effects on pro-inflammatory and anti-inflammatory cytokines. FIG. 2B shows an unbalanced state resulting from under-expression of TIMPs, permitting excessive MMP, ADAM, and ADAMTS activity, which is exacerbated by a positive feedback loop.

### DETAILED DESCRIPTION OF THE INVENTION

The present invention relates to a composition for treating an inflammatory and/or degenerative process in a human or other animal. The composition includes four or more (e.g., five or more, six or more, seven or more, or all eight) of the following: a MMP 1 inhibitor, a MMP 2 inhibitor, a MMP 3 inhibitor, a MMP 7 inhibitor, a MMP 9 inhibitor, an ADAMTS-4 inhibitor, a MMP 13 inhibitor, and a MMP 14 inhibitor.

In one embodiment, the composition includes a MMP 1 inhibitor. In another embodiment, the composition includes a MMP 2 inhibitor. In still another embodiment, the composition includes a MMP 3 inhibitor. In yet another embodiment, the composition includes a MMP 7 inhibitor. In still another embodiment, the composition includes a MMP 9 inhibitor. In yet another embodiment, the composition includes an ADAMTS-4 inhibitor. In still another embodiment, the composition includes a MMP 13 inhibitor. In yet another embodiment, the composition includes a MMP 14 inhibitor. In still another embodiment, the composition includes a MMP 3 inhibitor, a MMP9 inhibitor, an ADAMTS-4 inhibitor, and a MMP 13 inhibitor. In yet another embodiment, the composition includes a MMP 1 inhibitor, a MMP 3 inhibitor, a MMP9 inhibitor, an ADAMTS-4 inhibitor, and a MMP 13 inhibitor. In still another embodiment, the composition includes a MMP 1 inhibitor, a MMP 2 inhibitor, a MMP 3 inhibitor, a MMP 7 inhibitor, a MMP9 inhibitor, an ADAMTS-4 inhibitor, a MMP 13 inhibitor, and a MMP 14 inhibitor.

As used herein, “MMP 1 inhibitor” is meant to refer to any compound or combination of compounds that inhibit the activity of MMP 1. “MMP 1”, as used herein, is meant to refer to interstitial collagenase (also known as matrix metalloproteinase 1). Examples of MMP 1 inhibitors include polymethoxylated flavones, catechins, and combinations of polymethoxylated flavones and catechins. In one embodiment, the composition of the present invention contains a polymethoxylated flavone and a catechin, which, in combination, serve as an inhibitor of MMP 1.

As used herein, “MMP 2 inhibitor” is meant to refer to any compound or combination of compounds that inhibit the activity of MMP 2. “MMP 2”, as used herein, is meant to refer to gelatinase A (also known as matrix metalloproteinase 2). Examples of MMP 2 inhibitors include catechins. In one embodiment, the composition of the present invention contains a catechin, which serves as an inhibitor of MMP 2.

As used herein, “MMP 3 inhibitor” is meant to refer to any compound or combination of compounds that inhibit the activity of MMP 3. “MMP 3”, as used herein, is meant to refer to stromelysin-1 (also known as matrix metalloproteinase 3). Examples of MMP 3 inhibitors include curcuminoids, polymethoxylated flavones, boswellic acids, and combinations of curcuminoids, polymethoxylated flavones, and/or boswellic acids. In one embodiment, the composition of the present invention contains a curcuminoid, a polymethoxylated flavone, and a boswellic acid, which, in combination, serve as an inhibitor of MMP 3.

As used herein, “MMP 7 inhibitor” is meant to refer to any compound or combination of compounds that inhibit the activity of MMP 7. “MMP 7”, as used herein, is meant to refer to matrilysin (also known as matrix metalloproteinase 7). Examples of MMP 7 inhibitors include catechins. In one embodiment, the composition of the present invention contains a catechin, which serves as an inhibitor of MMP 7.

As used herein, “MMP 9 inhibitor” is meant to refer to any compound or combination of compounds that inhibit the activity of MMP 9. “MMP 9”, as used herein, is meant to refer to gelatinase B (also known as matrix metalloproteinase 9). Examples of MMP 9 inhibitors include curcuminoids, polymethoxylated flavones, catechins, and combinations of curcuminoids, polymethoxylated flavones, and/or catechins. In one embodiment, the composition of the present invention contains a curcuminoid, a polymethoxylated flavone, and a catechin, which, in combination, serve as an inhibitor of MMP 9.

As used herein, “ADAMTS-4 inhibitor” is meant to refer to any compound or combination of compounds that inhibit the activity of ADAMTS-4. “ADAMTS-4”, as used herein, is meant to refer to aggrecanase (a disintegrin and metalloproteinase with a thrombospondin motif). Examples of ADAMTS-4 inhibitors include curcuminoids, boswellic acids, and combinations of curcuminoids and/or boswellic acids. In one embodiment, the composition of the present invention contains a curcuminoid and a boswellic acid, which, in combination, serve as an inhibitor of ADAMTS-4.

As used herein, “MMP 13 inhibitor” is meant to refer to any compound or combination of compounds that inhibit the activity of MMP 13. “MMP 13”, as used herein, is meant to refer to collagenase 3 (also known as matrix metalloproteinase 13). Examples of MMP 13 inhibitors include curcuminoids, catechins, boswellic acids, and combinations of curcuminoids, catechins, and/or boswellic acids. In one embodiment, the composition of the present invention contains a curcuminoid, a catechin, and a boswellic acid, which, in combination, serve as an inhibitor of MMP 13.

As used herein, “MMP 14 inhibitor” is meant to refer to any compound or combination of compounds that inhibit the activity of MMP 14. “MMP 14”, as used herein, is meant to refer to membrane type 1-matrix metalloproteinase (also known as MT1-MMP and as matrix metalloproteinase 14). Examples of MMP 14 inhibitors include catechins. In one embodiment, the composition of the present invention contains a catechin, which serves as an inhibitor of MMP 14.

The various inhibitors included in the composition of the present invention can be specific for a particular enzyme (e.g., specific for MMP 1, MMP 2, MMP 3, MMP 7, MMP 9, ADAMTS-4, MMP 13, or MMP 14). Alternatively, non-specific inhibitors can be used (i.e., inhibitors that inhibit two or more (e.g., exactly two, exactly three, exactly four, exactly five, exactly six, three or more, four or more, and/or five or more) of MMP 1, MMP 2, MMP 3, MMP 7, MMP 9, ADAMTS-4, MMP 13, and MMP 14). Thus, for example, the composition of the present invention can include catechins to serve as MMP 1 inhibitors, as MMP 2 inhibitors, as MMP 7 inhibitors, as MMP 9 inhibitors, as MMP 13 inhibitors, and/or as MMP 14 inhibitors. As further illustration, the composition of the present invention can include polymethoxylated flavones to serve as MMP 1 inhibitors, as MMP 3 inhibitors, and/or as MMP 9 inhibitors. As yet further illustration, the composition of the present invention can include boswellic acids to serve as MMP 3 inhibitors, as ADAMTS-4 inhibitors, and/or as MMP 13 inhibitors. As still further illustration, the composition of the present invention can include curcuminoids to serve as MMP 3 inhibitors, as MMP 9 inhibitors, as ADAMTS-4 inhibitors, and/or as MMP 13 inhibitors.

In one embodiment, the composition of the present invention contains at least one inhibitor that inhibits two or more (e.g., exactly two, exactly three, exactly four, exactly five, exactly six, three or more, four or more, and/or five or more) of MMP 1, MMP 2, MMP 3, MMP 7, MMP 9, ADAMTS-4, MMP 13, and MMP 14. In another embodiment, the composition of the present invention contains at least two inhibitors, each of which inhibits two or more (e.g., exactly two, exactly three, exactly four, exactly five, exactly six, three or more, four or more, and/or five or more) of MMP 1, MMP 2, MMP 3, MMP 7, MMP 9, ADAMTS-4, MMP 13, and MMP 14. In still another embodiment, the composition of the present invention contains at least three inhibitors, each of which inhibits two or more (e.g., exactly two, exactly three, exactly four, exactly five, exactly six, three or more, four or more, and/or five or more) of MMP 1, MMP 2, MMP 3, MMP 7, MMP 9, ADAMTS-4, MMP 13, and MMP 14.

As will be evident from the above discussion, the composition of the present invention can employ more than one (e.g., more than two) inhibitors that inhibit MMP 1, MMP 2, MMP 3, MMP 7, MMP 9, ADAMTS-4, MMP 13, and/or MMP 14. For example, inhibition of MMP 1 can be achieved using polymethoxylated flavones and catechins. As further illustration, inhibition of MMP 3 can be achieved using polymethoxylated flavones and curcuminoids; polymethoxylated flavones and boswellic acids; curcuminoids and boswellic acids; or polymethoxylated flavones, curcuminoids, and boswellic acids. As still further illustration, inhibition of MMP 9 can be achieved using polymethoxylated flavones and curcuminoids; polymethoxylated flavones and catechins; curcuminoids and catechins; or polymethoxylated flavones, curcuminoids, and catechins. As yet further illustration, inhibition of ADAMTS-4 can be achieved using curcuminoids and boswellic acids. As still further illustration, inhibition of MMP 13 can be achieved using boswellic acids and curcuminoids; boswellic acids and catechins; curcuminoids and catechins; or boswellic acids, curcuminoids, and catechins.

It is to be understood that the term “inhibit” and the terms “inhibition”, “inhibitor”, “inhibiting”, and other forms of the word “inhibit”, as used herein in regards to the enzymes described in the present application (e.g., MMP 1, MMP 2, MMP 3, MMP 7, MMP 9, ADAMTS-4, MMP 13, and/or MMP 14) are meant to refer any mechanism by which the activity of the enzyme is reduced, such as in those cases where the enzyme is directly inhibited as well as those cases where the enzyme is indirectly inhibited. For example, these terms are meant to include those situations in which the enzyme's activity is reduced by interfering with the production of the enzyme. As further illustration, these terms are also meant to include those situations in which the enzyme's activity is reduced by promoting the degradation of the enzyme.

Inhibition of the enzymes described in the present application (e.g., MMP 1, MMP 2, MMP 3, MMP 7, MMP 9, ADAMTS-4, MMP 13, and/or MMP 14) can be achieved by reducing their production in a variety of ways.

For example, MMP production can be reduced by suppressing Transcription Factor AP1. Transcription Factor AP1 can be suppressed, for example, with curcuminoids and/or with catechins.

As further illustration, MMP production can be reduced by suppressing Transcription Factor c-Jun. Transcription Factor c-Jun can be suppressed, for example, with catechins.

As yet further illustration, MMP production can be reduced by suppressing Transcription Factor NFκB. Transcription Factor NFκB can be suppressed, for example, by suppressing SAPK/JNK (stress-activated protein kinase/c-Jun N-terminal kinase), such as with curcuminoids); by suppressing IκBα kinase phosphorylation (e.g., with curcuminoids); by suppressing IL1β gene expression (e.g., with polymethoxylated flavones and/or catechins); by suppressing ERK1/2 (e.g., with boswellic acids and/or catechins); by suppressing p38 MAPK (e.g., with catechins); by suppressing TNFα gene expression (e.g., with polymethoxylated flavones); and/or by suppressing IL1α gene expression (e.g., with polymethoxylated flavones). Alternatively or additionally, Transcription Factor NFκB can be suppressed by suppressing ProTNF activation to TNFα, for example by MMP 1, 2, 3, 7, 9, 13, and/or 14 or by TACE inhibition (e.g., via TIMP) (e.g., with polymethoxylated flavones and/or with a Trypterigium wilfordii Hook extract). In should also be noted that in addition to reducing MMP production, suppression of some of the aforesaid processes can have other beneficial effects. For example, suppression of SAPK/JNK and suppression of IκBα kinase phosphorylation can result in a reduction in TNFα production, and suppression of ERK1/2 can result in a reduction in ADAMTS-4 and other ADAMTS production.

As still further illustration, MMP production can be reduced by suppressing MMP gene expression, for example, with polymethoxylated flavones and/or with a Trypterigium wilfordii Hook extract.

As yet further illustration, MMP production can be reduced by suppressing ProMMP2 activation by MT1-MMP.

As still further illustration, MMP production can be reduced by upregulating TIMPH2 (e.g., with Trypterigium wilfordii Hook extract) or by upregulating TIMP1 (e.g., with polymethoxylated flavones and/or with a Trypterigium wilfordii Hook extract). In addition to reducing MMP production, upregulation of TIMPH2 and/or TIMP1 can also result in a reduction in TNFα production.

The aforementioned inhibitors of MMP (e.g., curcuminoids, catechins, polymethoxylated flavones, boswellic acids, and Trypterigium wilfordii Hook extracts) can also affect other chemical processes that may contribute to or exacerbate inflammatory and/or degenerative processes. Illustratively, Trypterigium wilfordii Hook extracts can also serve to suppress IL6 gene expression, which, in turn, can result in a decrease in macrophage and monocyte activation. As further illustration, curcuminoids, catechins, polymethoxylated flavones, boswellic acids, and/or Trypterigium wilfordii Hook extracts can also serve to suppress cytokine upregulation (e.g., by suppressing gene expression of IL1α, IL1β, TNFα, and/or IL6 and/or by suppressing MMP activation of TNFα), which, in turn, can result in a decrease in cytokines. As yet further illustration, curcuminoids can also serve to suppress release of hydrolases and eicosanoids by macrophages, which, in turn, can result in a decrease in acute phase responses and in a reduction in hydrolysis.

Inhibition of the enzymes described in the present application (e.g., MMP 1, MMP 2, MMP 3, MMP 7, MMP 9, ADAMTS-4, MMP 13, and/or MMP 14), for example, by reducing MMP production can have a number of consequences that are beneficial to the treatment of inflammatory and/or degenerative processes. Illustratively, inhibition of the enzymes described in the present application can result in the suppression of chemokine upregulation. For example, suppression of chemokine upregulation via MMP activation of fractalkine can result in decreased leukocyte chemo-attraction; suppression of chemokine upregulation via MMP7 activation of KC (a chemokine CXCL1) can result in decreased leukocyte chemo-attraction; suppression of chemokine upregulation by a reduction in TNFα can result in decreased neutrophil attraction; suppression of chemokine upregulation by a reduction in MMP (e.g., MMP 2, 3, 7, and/or 9) activation of TGFβ can result in decreased macrophage and monocyte chemo-attraction as well as in decreased IL1 synthesis by macrophages; and suppression of chemokine upregulation by a reduction in MMP9 upregulation of IL8 can result in a reduction in macrophages.

By interfering with the aforementioned biochemical processes, enzymes described in the present application (e.g., MMP 1, MMP 2, MMP 3, MMP 7, MMP 9, ADAMTS-4, MMP 13, and/or MMP 14) can be inhibited by decreasing the production of the MMPs and ADAMTS-4. As noted above, interference with the aforementioned biochemical processes can involve direct interference with MMP and ADAMTS-4 production by suppressing chemical messengers involved in transcription factor activation, or interference with the aforementioned biochemical processes can involve indirect interference with MMP and ADAMTS-4 production by suppressing upstream mediators of their production (e.g., the interleukins and TNFα).

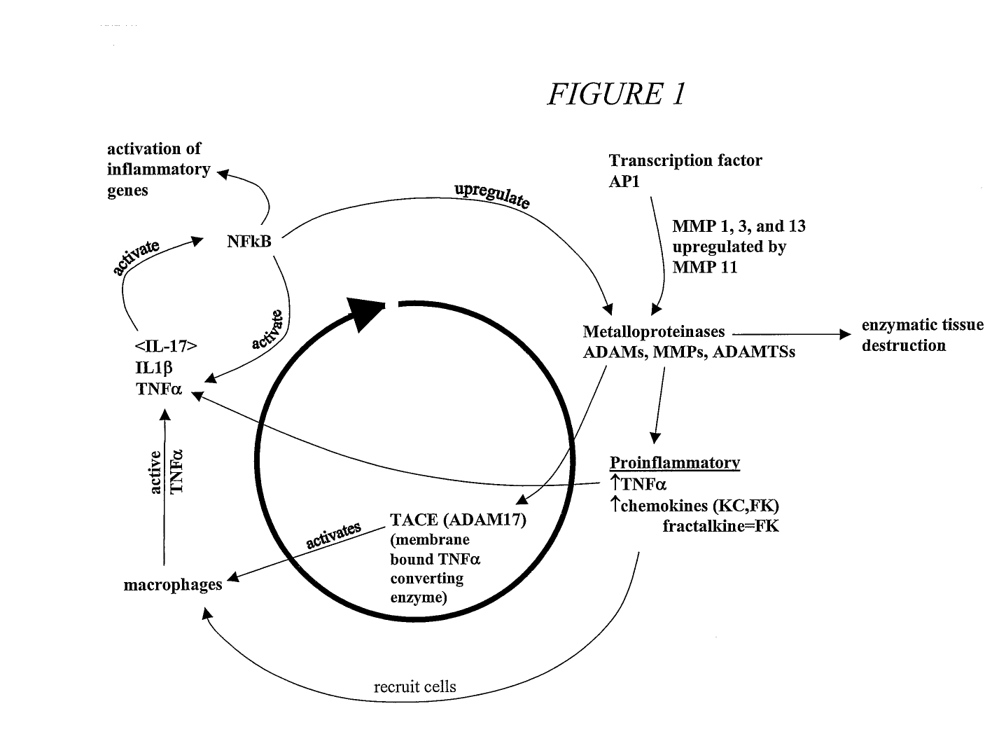

It is to be understood that the mechanism by which inhibition of the enzymes described in the present application (e.g., MMP 1, MMP 2, MMP 3, MMP 7, MMP 9, ADAMTS-4, MMP 13, and/or MMP 14) results in efficacious treatment of inflammatory and/or degenerative processes is not particularly critical to the practice of the present invention, and the nature of such mechanisms are not to be construed in any way as limitations on the methods and compositions of the present invention. Applicant believes that the compositions of the present invention are effective because MMP and ADAMTS-4 inhibition have a number of downstream effects. It is believed that MMPs play a central role in a cycle involving MMP activation of cytokines and chemokines followed by chemokines and cytokines upregulating the production of more MMPs. An imbalance of MMPs and their natural inhibitors (e.g., TIMPs) leads to an unregulated cycle of increased cytokine/chemokine/MMP production, for example, as illustrated in FIG. 1. The result of this unregulated cascade of biochemical events is chronic, progressive degradation of tissue (by MMPs) and chronic inflammation of tissue (by chemokines and cytokines). Inhibition of this excessive production of MMPs will result in decreased MMP substrate degradation (e.g., collagen, aggrecan, proteoglycan link protein, gelatin, elastin, fibronectin, versican, laminin, vitronectin, entactin, dermatan sulfate proteogycan, nidogen, tenascin, amelogenin, casein, α1 proteinase inhibitor, etc.); decreased cytokine and chemokine production (e.g., TNFα, syndecan 1, KC, fractalkine (“KF”), TGFβ, IL1 by macrophage, IL8 by MMP 9, etc.); and/or decreased self activation, such as that resulting from MMPs acting on proMMPs. It is believed that the various components in the composition of the present invention (i.e., MMP 1, MMP 2, MMP 3, MMP 7, MMP 9, ADAMTS-4, MMP 13, and/or MMP 14 inhibitors) affect a variety of different biochemical processes involved in the synthesis and activation of various MMPs. It is further believed that this results in a broad spectrum of biochemical processes being influenced, as well as a broad spectrum of MMPs being inhibited by these processes. This spectrum of MMPs has been targeted for inhibition, not only because of their tissue degradative properties, but also because of their role in the activation (e.g., by cleavage) of other substrates likely to be involved in the initiation and propagation of a chronic, self-perpetuating cycle of chronic inflammation.

The present invention, in another aspect thereof, relates to a composition for treating an inflammatory and/or degenerative process in a human or other animal, wherein the composition includes a curcuminoid, a polymethoxylated flavone, a catechin, and a boswellic acid.

As used herein, “curcuminoid” is meant to refer to one or more of the polyphenolic pigments found in the spice turmeric and/or in the plant Curcuma longa L., especially in the rhizomes of the plant. “Curcuminoid”, as used herein, is meant to include, for example, curcumin, demethoxycurcumin, and bisdemethoxycurcumin, as well as combinations of curcumin, demethoxycurcumin, and bisdemethoxycurcumin. The curcuminoids used in the composition of the present invention can be prepared by extracting tumeric with an alcohol (e.g., ethanol). They can be purified to any suitable level, such as about 50%, about 60%, about 70%, about 80%, about 85%, about 90%, and/or about 95%, for example, by repeated extraction. One example of a suitable curcuminoid is an extract (e.g., a 98% extract) of tetrahydrocurcumin.

As used herein, “polymethoxylated flavone” is meant to refer to flavonoids in which hydroxyl groups are replaced with methoxy groups. Examples of polymethoxylated flavones include tangeretin and nobiletin, both of which are concentrated in the peel of citrus fruits. The polymethoxylated flavones used in the composition of the present invention can be prepared by extracting one of more polymethoxylated flavones from citrus peel. They can be purified to any suitable level, such as about 50%, about 60%, about 70%, about 80%, about 85%, about 90%, and/or about 95%, for example, by repeated extraction. One example of a suitable polymethoxylated flavone is an extract (e.g., a 5:1 extract) of Citrus reticulata peel.

As used herein, “catechin” is meant to refer to flavonoid phytochemical compounds that appear predominantly in green tea and, to a lesser extent, in black tea, grapes, wine, and chocolate. Examples of catechins that can be used in the practice of the present invention include gallocatechin (“GC”), epigallocatechin (“EGC”), epicatechin (“EC”), epicatechin gallate (“ECG”), and epigallocatechin gallate (“EGCG”), as well as mixtures of these and other catechins. The catechins used in the composition of the present invention can be prepared from lipid extracts from green tea leaves. The catechins can be purified to any suitable level, such as about 50%, about 60%, about 70%, about 80%, about 85%, about 90%, and/or about 95%. One example of a suitable catechin is an extract (e.g., an 80% catechin extract) of Camellia sinensis.

As used herein, “boswellic acid” is meant to include β-boswellic acid, acetyl-β-boswellic acid, 11-keto-β-boswellic acid, lower alkyl esters of 11-keto-β-boswellic acid (e.g., acetyl-11-keto-β-boswellic acid), α-boswellic acid, and γ-boswellic acid, as well as mixtures of these and other boswellic acids. The boswellic acids can be obtained from plants that contain these compounds, such as Boswellia (serrata, papyrifera, frereana, carteri, thurifera, glabra, bhaw-dajiana, oblongata, socotrana and other members of this family). Illustratively, boswellic acids can be obtained by ethanol extraction from the gum of Boswellia serrata. The boswellic acids can be purified to any suitable level, such as about 50%, about 60%, about 70%, about 80%, about 85%, about 90%, and/or about 95%.

The compositions of the present invention can also include additional components. Illustratively, the compositions of the present invention can also include a Harapagophytum procumbens extract, a Trypterigium wilfordii Hook extract, a Glycyrrhiza glabra extract, a Cinnamomum cassia extract, a Magnolia obovata extract, a Magnolia officianalis extract, and/or a Euonymus alatus extract. In one illustrative embodiment, the compositions of the present invention further include a Harapagophytum procumbens extract. In another illustrative embodiment, the compositions of the present invention further include a Trypterigium wilfordii Hook extract. In yet another illustrative embodiment, the compositions of the present invention further include a Glycyrrhiza glabra extract. In still another illustrative embodiment, the compositions of the present invention further include a Cinnamomum cassia extract. In still another illustrative embodiment, the compositions of the present invention further include a Magnolia obovata extract. In still another illustrative embodiment, the compositions of the present invention further include a Magnolia officianalis extract. In yet another illustrative embodiment, the compositions of the present invention further include a Euonymus alatus extract.

The compositions of the present invention can also include two or more of the aforementioned extracts. For example, in one illustrative embodiment, the compositions of the present invention further include a Trypterigium wilfordii Hook extract and a Glycyrrhiza glabra extract. In another illustrative embodiment, the compositions of the present invention further include a Trypterigium wilfordii Hook extract and a Cinnamomum cassia extract. In still another illustrative embodiment, the compositions of the present invention further include a Trypterigium wilfordii Hook extract and a Magnolia obovata extract. In still another illustrative embodiment, the compositions of the present invention further include a Trypterigium wilfordii Hook extract and a Magnolia officianalis extract. In yet another illustrative embodiment, the compositions of the present invention further include a Trypterigium wilfordii Hook extract and a Euonymus alatus extract. In yet another illustrative embodiment, the compositions of the present invention further include a Glycyrrhiza glabra extract and a Cinnamomum cassia extract. In still another illustrative embodiment, the compositions of the present invention further include a Glycyrrhiza glabra extract and a Magnolia obovata extract. In still another illustrative embodiment, the compositions of the present invention further include a Glycyrrhiza glabra extract and a Magnolia officianalis extract. In yet another illustrative embodiment, the compositions of the present invention further include a Glycyrrhiza glabra extract and a Euonymus alatus extract. In still another illustrative embodiment, the compositions of the present invention further include a Cinnamomum cassia extract and a Magnolia obovata extract. In still another illustrative embodiment, the compositions of the present invention further include a Cinnamomum cassia extract and a Magnolia officianalis extract. In yet another illustrative embodiment, the compositions of the present invention further include a Cinnamomum cassia extract and a Euonymus alatus extract. In still another illustrative embodiment, the compositions of the present invention further include a Magnolia obovata extract and a Magnolia officianalis extract. In yet another illustrative embodiment, the compositions of the present invention further include a Magnolia obovata extract and a Euonymus alatus extract. In yet another illustrative embodiment, the compositions of the present invention further include a Magnolia officianalis extract and a Euonymus alatus extract. In still other illustrative embodiments, the compositions of the present invention further include a Harapagophytum procumbens extract and a second extract selected from the group consisting of a Trypterigium wilfordii Hook extract, a Glycyrrhiza glabra extract, a Cinnamomum cassia extract, a Magnolia obovata extract, a Magnolia officianalis extract, and a Euonymus alatus extract.

The compositions of the present invention can also include three or more of the aforementioned extracts. For example, in one illustrative embodiment, the compositions of the present invention further include a Trypterigium wilfordii Hook extract, a Glycyrrhiza glabra extract, and a Cinnamomum cassia extract. In another illustrative embodiment, the compositions of the present invention further include a Trypterigium wilfordii Hook extract, a Glycyrrhiza glabra extract, and a Magnolia obovata extract. In still another illustrative embodiment, the compositions of the present invention further include a Trypterigium wilfordii Hook extract, a Glycyrrhiza glabra extract, and a Magnolia officianalis extract. In yet another illustrative embodiment, the compositions of the present invention further include a Trypterigium wilfordii Hook extract, a Glycyrrhiza glabra extract, and a Euonymus alatus extract. In still another illustrative embodiment, the compositions of the present invention further include a Trypterigium wilfordii Hook extract, a Cinnamomum cassia extract, and a Magnolia obovata extract. In yet another illustrative embodiment, the compositions of the present invention further include a Trypterigium wilfordii Hook extract, a Cinnamomum cassia extract, and a Magnolia officianalis extract. In still another illustrative embodiment, the compositions of the present invention further include a Trypterigium wilfordii Hook extract, a Cinnamomum cassia extract, and a Euonymus alatus extract. In still another illustrative embodiment, the compositions of the present invention further include a Trypterigium wilfordii Hook extract, a Magnolia obovata extract, and a Magnolia officianalis extract. In still another illustrative embodiment, the compositions of the present invention further include a Trypterigium wilfordii Hook extract, a Magnolia obovata extract, and a Euonymus alatus extract. In yet another illustrative embodiment, the compositions of the present invention further include a Trypterigium wilfordii Hook extract, a Magnolia officianalis extract, and a Euonymus alatus extract. In yet another illustrative embodiment, the compositions of the present invention further include a Glycyrrhiza glabra extract, a Cinnamomum cassia extract, and a Magnolia obovata extract. In still another illustrative embodiment, the compositions of the present invention further include a Glycyrrhiza glabra extract, a Cinnamomum cassia extract, and a Magnolia officianalis extract. In yet another illustrative embodiment, the compositions of the present invention further include a Glycyrrhiza glabra extract, a Cinnamomum cassia extract, and a Euonymus alatus extract. In still another illustrative embodiment, the compositions of the present invention further include a Glycyrrhiza glabra extract, a Magnolia obovata extract, and a Magnolia officianalis extract. In yet another illustrative embodiment, the compositions of the present invention further include a Glycyrrhiza glabra extract, a Magnolia obovata extract, and a Euonymus alatus extract. In still another illustrative embodiment, the compositions of the present invention further include a Glycyrrhiza glabra extract, a Magnolia officianalis extract, and a Euonymus alatus extract. In yet another illustrative embodiment, the compositions of the present invention further include a Cinnamomum cassia extract, and a Magnolia obovata extract, and a Magnolia officianalis extract. In still another illustrative embodiment, the compositions of the present invention further include a Cinnamomum cassia extract, a Magnolia obovata extract, and a Euonymus alatus extract. In yet another illustrative embodiment, the compositions of the present invention further include a Cinnamomum cassia extract, a Magnolia officianalis extract, and a Euonymus alatus extract. In still another illustrative embodiment, the compositions of the present invention further include a Magnolia obovata extract, a Magnolia officianalis extract, and a Euonymus alatus extract. In still other illustrative embodiments, the compositions of the present invention further include a Harapagophytum procumbens extract and two additional extracts selected from the group consisting of a Trypterigium wilfordii Hook extract, a Glycyrrhiza glabra extract, a Cinnamomum cassia extract, a Magnolia obovata extract, a Magnolia officianalis extract, and a Euonymus alatus extract.

The compositions of the present invention can also include four or more of the aforementioned extracts. For example, in one illustrative embodiment, the compositions of the present invention further include a Trypterigium wilfordii Hook extract, a Glycyrrhiza glabra extract, a Cinnamomum cassia extract, and a Magnolia obovata extract. In another illustrative embodiment, the compositions of the present invention further include a Trypterigium wilfordii Hook extract, a Glycyrrhiza glabra extract, a Cinnamomum cassia extract, and a Magnolia officianalis extract. In still another illustrative embodiment, the compositions of the present invention further include a Trypterigium wilfordii Hook extract, a Glycyrrhiza glabra extract, a Cinnamomum cassia extract, and a Euonymus alatus extract. In yet another illustrative embodiment, the compositions of the present invention further include a Trypterigium wilfordii Hook extract, a Glycyrrhiza glabra extract, a Magnolia obovata extract, and a Magnolia officianalis extract. In yet another illustrative embodiment, the compositions of the present invention further include a Trypterigium wilfordii Hook extract, a Glycyrrhiza glabra extract, a Magnolia obovata extract, and a Euonymus alatus extract. In still another illustrative embodiment, the compositions of the present invention further include a Trypterigium wilfordii Hook extract, a Glycyrrhiza glabra extract, a Magnolia officianalis extract, and a Euonymus alatus extract. In yet another illustrative embodiment, the compositions of the present invention further include a Trypterigium wilfordii Hook extract, a Cinnamomum cassia extract, a Magnolia obovata extract, and a Magnolia officianalis extract. In yet another illustrative embodiment, the compositions of the present invention further include a Trypterigium wilfordii Hook extract, a Cinnamomum cassia extract, a Magnolia obovata extract, and a Euonymus alatus extract. In still another illustrative embodiment, the compositions of the present invention further include a Trypterigium wilfordii Hook extract, a Cinnamomum cassia extract, a Magnolia officianalis extract, and a Euonymus alatus extract. In yet another illustrative embodiment, the compositions of the present invention further include a Trypterigium wilfordii Hook extract, a Magnolia obovata extract, a Magnolia officianalis extract, and a Euonymus alatus extract. In still another illustrative embodiment, the compositions of the present invention further include a Glycyrrhiza glabra extract, a Cinnamomum cassia extract, a Magnolia obovata extract, and a Magnolia officianalis extract. In yet another illustrative embodiment, the compositions of the present invention further include a Glycyrrhiza glabra extract, a Cinnamomum cassia extract, a Magnolia obovata extract, and a Euonymus alatus extract. In still another illustrative embodiment, the compositions of the present invention further include a Glycyrrhiza glabra extract, a Cinnamomum cassia extract, a Magnolia officianalis extract, and a Euonymus alatus extract. In yet another illustrative embodiment, the compositions of the present invention further include a Glycyrrhiza glabra extract, a Magnolia obovata extract, a Magnolia officianalis extract, and a Euonymus alatus extract. In still another illustrative embodiment, the compositions of the present invention further include a Cinnamomum cassia extract, a Magnolia obovata extract, a Magnolia officianalis extract, and a Euonymus alatus extract. In still other illustrative embodiments, the compositions of the present invention further include a Harapagophytum procumbens extract and three additional extracts selected from the group consisting of a Trypterigium wilfordii Hook extract, a Glycyrrhiza glabra extract, a Cinnamomum cassia extract, a Magnolia obovata extract, a Magnolia officianalis extract, and a Euonymus alatus extract.

The compositions of the present invention can also include five or more of the aforementioned extracts. For example, in one illustrative embodiment, the compositions of the present invention further include a Trypterigium wilfordii Hook extract, a Glycyrrhiza glabra extract, a Cinnamomum cassia extract, a Magnolia obovata extract, and a Magnolia officianalis extract. In another illustrative embodiment, the compositions of the present invention further include a Trypterigium wilfordii Hook extract, a Glycyrrhiza glabra extract, a Cinnamomum cassia extract, a Magnolia obovata extract, and a Euonymus alatus extract. In still another illustrative embodiment, the compositions of the present invention further include a Trypterigium wilfordii Hook extract, a Glycyrrhiza glabra extract, a Cinnamomum cassia extract, a Magnolia officianalis extract, and a Euonymus alatus extract. In yet another illustrative embodiment, the compositions of the present invention further include a Trypterigium wilfordii Hook extract, a Glycyrrhiza glabra extract, a Magnolia obovata extract, a Magnolia officianalis extract, and a Euonymus alatus extract. In still another illustrative embodiment, the compositions of the present invention further include a Trypterigium wilfordii Hook extract, a Cinnamomum cassia extract, a Magnolia obovata extract, a Magnolia officianalis extract, and a Euonymus alatus extract. In yet another illustrative embodiment, the compositions of the present invention further include a Glycyrrhiza glabra extract, a Cinnamomum cassia extract, a Magnolia obovata extract, a Magnolia officianalis extract, and a Euonymus alatus extract. In still other illustrative embodiments, the compositions of the present invention further include a Harapagophytum procumbens extract and four additional extracts selected from the group consisting of a Trypterigium wilfordii Hook extract, a Glycyrrhiza glabra extract, a Cinnamomum cassia extract, a Magnolia obovata extract, a Magnolia officianalis extract, and a Euonymus alatus extract.

The compositions of the present invention can also include six or more of the aforementioned extracts. For example, in one illustrative embodiment, the compositions of the present invention further include a Harapagophytum procumbens extract, a Trypterigium wilfordii Hook extract, a Glycyrrhiza glabra extract, a Cinnamomum cassia extract, a Magnolia obovata extract, and a Magnolia officianalis extract. In another illustrative embodiment, the compositions of the present invention further include a Harapagophytum procumbens extract, a Trypterigium wilfordii Hook extract, a Glycyrrhiza glabra extract, a Cinnamomum cassia extract, a Magnolia obovata extract, and a Euonymus alatus extract. In still another illustrative embodiment, the compositions of the present invention further include a Harapagophytum procumbens extract, a Trypterigium wilfordii Hook extract, a Glycyrrhiza glabra extract, a Cinnamomum cassia extract, a Magnolia officianalis extract, and a Euonymus alatus extract. In yet another illustrative embodiment, the compositions of the present invention further include a Harapagophytum procumbens extract, a Trypterigium wilfordii Hook extract, a Glycyrrhiza glabra extract, a Magnolia obovata extract, a Magnolia officianalis extract, and a Euonymus alatus extract. In still another illustrative embodiment, the compositions of the present invention further include a Harapagophytum procumbens extract, a Trypterigium wilfordii Hook extract, a Cinnamomum cassia extract, a Magnolia obovata extract, a Magnolia officianalis extract, and a Euonymus alatus extract. In yet another illustrative embodiment, the compositions of the present invention further include a Harapagophytum procumbens extract, a Glycyrrhiza glabra extract, a Cinnamomum cassia extract, a Magnolia obovata extract, a Magnolia officianalis extract, and a Euonymus alatus extract. In still another illustrative embodiment, the compositions of the present invention further include a Trypterigium wilfordii Hook extract, a Glycyrrhiza glabra extract, a Cinnamomum cassia extract, a Magnolia obovata extract, a Magnolia officianalis extract, and a Euonymus alatus extract.

The compositions of the present invention can also include all of the aforementioned extracts. For example, in one illustrative embodiment, the compositions of the present invention further include a Harapagophytum procumbens extract, a Trypterigium wilfordii Hook extract, a Glycyrrhiza glabra extract, a Cinnamomum cassia extract, a Magnolia obovata extract, a Magnolia officianalis extract, and a Euonymus alatus extract.

It is believed that compositions of the present invention that further include (i.e., in addition to the aforementioned curcuminoids, polymethoxylated flavones, and boswellic acids) a Harapagophytum procumbens extract, a Trypterigium wilfordii Hook extract, a Glycyrrhiza glabra extract, a Cinnamomum cassia extract, a Magnolia obovata extract, a Magnolia officianalis extract, and/or a Euonymus alatus extract may provide additional benefits in the treatment of inflammatory and/or degenerative processes. In this regard, it is to be understood that the mechanism by which these additional components (i.e., the Trypterigium wilfordii Hook extract, the Glycyrrhiza glabra extract, the Cinnamomum cassia extract, the Magnolia obovata extract, the Magnolia officianalis extract, and/or the Euonymus alatus extract) act is not particularly critical to the practice of the present invention, and the nature of such mechanisms are not to be construed in any way as limitations on the methods and compositions of the present invention. Applicant believes that these additional components are effective because they can further inhibit one or more of the various enzymes discussed above (e.g., one or more of MMP 1, MMP 2, MMP 3, MMP 7, MMP 9, MMP 13, and MMP 14).

The compositions of the present invention can be formulated so as to contain from about 0.01 wt % to about 50 wt % (e.g., from about 0.1 wt % to about 25 wt % and/or from about 1 wt % to about 20 wt %) of curcuminoids. The compositions of the present invention can be formulated so as to contain from about 0.01 wt % to about 50 wt % (e.g., from about 0.1 wt % to about 25 wt % and/or from about 1 wt % to about 20 wt %) of polymethoxylated flavones. The compositions of the present invention can be formulated so as to contain from about 0.01 wt % to about 50 wt % (e.g., from about 0.1 wt % to about 25 wt % and/or from about 1 wt % to about 20 wt %) of catechins. The compositions of the present invention can be formulated so as to contain from about 0.01 wt % to about 50 wt % (e.g., from about 0.1 wt % to about 25 wt % and/or from about 1 wt % to about 20 wt %) of boswellic acids. The compositions of the present invention can be formulated such that the weight ratio of curcuminoids to polymethoxylated flavones is from about 50:1 to about 1:50, such as from about 20:1 to about 1:20 and/or from about 10:1 to about 1:10. Additionally or alternatively, the compositions of the present invention can be formulated such that the weight ratio of curcuminoids to catechins is from about 50:1 to about 1:50, such as from about 20:1 to about 1:20 and/or from about 10:1 to about 1:10. Still additionally or alternatively, the compositions of the present invention can be formulated such that the weight ratio of curcuminoids to boswellic acids is from about 50:1 to about 1:50, such as from about 20:1 to about 1:20 and/or from about 10:1 to about 1:10. Still additionally or alternatively, the compositions of the present invention can be formulated such that the weight ratio of polymethoxylated flavones to catechins is from about 50:1 to about 1:50, such as from about 20:1 to about 1:20 and/or from about 10:1 to about 1:10. Still additionally or alternatively, the compositions of the present invention can be formulated such that the weight ratio of polymethoxylated flavones to boswellic acids is from about 50:1 to about 1:50, such as from about 20:1 to about 1:20 and/or from about 10:1 to about 1:10. Still additionally or alternatively, the compositions of the present invention can be formulated such that the weight ratio of catechins to boswellic acids is from about 50:1 to about 1:50, such as from about 20:1 to about 1:20 and/or from about 10:1 to about 1:10. For example, the compositions of the present invention can be formulated such that the curcuminoid:polymethoxylated flavone:catechin:boswellic acid weight ratio is W:X:Y:Z, where each of W, X, Y, and Z independently represents a number between 1 and 50, inclusive, such as in the case where each of W, X, Y, and Z independently represents a number between 1 and 20, inclusive, and/or in the case where each of W, X, Y, and Z independently represents a number between 1 and 10, inclusive.

In cases where the compositions of the present invention contain one or more of the aforementioned Harapagophytum procumbens extract, Trypterigium wilfordii Hook extract, Glycyrrhiza glabra extract, Cinnamomum cassia extract, Magnolia obovata extract, Magnolia officianalis extract, and Euonymus alatus extract, the compositions can be formulated so as to contain from about 0.01 wt % to about 50 wt % (e.g., from about 0.1 wt % to about 25 wt % and/or from about 1 wt % to about 20 wt %) of such extracts, in the aggregate. Additionally or alternatively, the aforementioned Harapagophytum procumbens extract, Trypterigium wilfordii Hook extract, Glycyrrhiza glabra extract, Cinnamomum cassia extract, Magnolia obovata extract, Magnolia officianalis extract, and/or Euonymus alatus extract can be present in an aggregate weight that is from about 0.02 to about 50 times (e.g., from about 0.05 to about 20 times and/or from about 0.1 to about 10 times the sum of the weights of the curcuminoid, polymethoxylated flavone, catechin, and boswellic acid components.

The compositions can further include other materials depending on the manner in which the composition is to be used.

For example, in the case where the composition is in the form of a pill, the composition can further include various inert diluents, carriers, and excipients commonly used in the formulation of tablets, capsules, and other pill forms.

Capsules can be prepared by mixing the active ingredients (e.g., the curcuminoid, polymethoxylated flavone, catechin, and boswellic acid components and the optional extracts) with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders.

Tablets can be prepared by direct compression, by wet granulation, or by dry granulation. Their formulations usually incorporate diluents, binders, lubricants, and disintegrators (in addition to the active components, such as in addition to the curcuminoid, polymethoxylated flavone, catechin, and boswellic acid components and the optional extracts). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The compositions of the present invention (e.g., those containing the curcuminoid, polymethoxylated flavone, catechin, and boswellic acid components and the optional extracts) can also be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects.

A lubricant can be used in the tablet formulation to prevent the tablet and punches from sticking in the die. The lubricant can be chosen from such slippery solids as talc, magnesium and calcium stearate, stearic acid, and hydrogenated vegetable oils.

Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Pill forms can also be formulated as enteric formulations, for example, to protect one or more of the active ingredients from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

Alternatively, the composition can be in a liquid form, such as in the form of a dispersion, a suspension, a solution, a syrup, or an elixir. Such dispersions, suspensions, solutions, syrups, and elixirs may contain conventional excipients, for example, methyl cellulose, tragacanth, sodium alginate; wetting agents, such as lecithin and polyoxyethylene stearate; and preservatives, such as ethyl-p-hydroxybenzoate.

Still alternatively, the composition can be in a powder or granular form. Such powders and granules can include diluents, such as starch, lactose, mannitol, kaolin, calcium phosphate or sulfate, inorganic salts (such as sodium chloride), powdered sugar, and powdered cellulose derivatives. Binders can also be used in powder and granular formulations. Suitable binders include starch, gelatin, sugars (e.g., lactose, fructose, glucose, and the like), natural and synthetic gums (e.g., acacia, alginates, methylcellulose, polyvinylpyrrolidine), polyethylene glycol, ethylcellulose, and waxes.

The composition of the present invention can be in a dietary supplement form. As used herein, “dietary supplement” is meant to refer to compositions which, in addition to containing the active ingredients (e.g., the curcuminoid, polymethoxylated flavone, catechin, and boswellic acid components and the optional extracts), also contain one or more essential nutrients. As used herein, “essential nutrients” are those nutrients which are required to sustain health but which cannot be effectively produced by one or more animals or by humans. Examples of essential nutrients are compiled in a number of published sources, including Modern Nutrition in Health and Disease, 8th ed., Shils et al., eds., Philadelphia: Lea and Febiger (1994), which is hereby incorporated by reference. Essential nutrients are meant to include essential vitamins and provitamins thereof, essential fats, essential minerals, such as those minerals for which daily values have been recommended, and essential amino acids. One example of a dietary supplement is a formulation which contains a vitamin and a caloric content of less than 2.5 cal per dry gram, such as less than 2 cal per dry gram and/or less than 1.8 cal per dry gram. Dietary supplements also include those materials which contain at least one vitamin in an amount greater than 15%, such as greater than 20% and/or greater than 40% of the U.S. adult RDA for that essential nutrient per gram of the dietary supplement. Still other suitable dietary supplements contain at least two vitamins, each in an amount greater than 10%, preferably greater than 15%, more preferably greater than 20% of the U.S. adult RDA for that essential nutrient per gram of essential nutrient preparation. Suitable dietary supplements are commonly referred to as vitamin supplements, mineral supplements, multiple vitamin supplements, and the like. The dietary supplements can be in the form of pills (e.g., tablets or capsules), powders, granules, liquids (e.g., solutions, dispersions, suspensions, syrups, and elixirs), or other forms.

The composition of the present invention can be in a food preparation form. Food preparations are materials which contain one or more amino acid, carbohydrate, or fat, which are suitable for human or animal consumption, and which are not essential nutrient preparations. Examples of food preparations include, for example, juices, nectars, and purees of various fruits and vegetables; breads, cereals, and other food products containing grains, such as rye flour, wheat flour, oat bran, etc. Food preparations suitable for human consumption include breakfast foods, such as prepared cereals, toaster pastries, and breakfast drink mixes; complete diet formulas; and weight-loss preparations, such as weight-loss drinks and weight-loss bars. Food preparations are also meant to include animal feed, animal feed supplements, and pet foods.

It will be appreciated that the actual preferred concentration of active ingredients (e.g., the curcuminoid, polymethoxylated flavone, catechin, and boswellic acid components and the optional extracts) in the composition will vary according to the particular formulation of active ingredients, the form of the composition, and the customarily consumed quantity of the composition. Many factors that may modify the action of the active ingredients (e.g., species of the subject, sex of the subject, body weight of the subject, diet, time of administration, rate of excretion, condition of the subject, drug combinations, and reaction sensitivities and severities) can be taken into account by those skilled in the art. Administration can be carried out continuously or periodically within the maximum tolerated dose. Optimal administration rates for a given set of conditions can be ascertained by those skilled in the art using conventional dosage administration tests.

The compositions of the present invention can be used to treat inflammatory and/or degenerative processes in a human or other subject. “Subject”, as used herein, is meant to include humans, as well as non-human animals, particularly those who suffer from or who are susceptible to developing inflammatory and/or degenerative diseases, inflammatory and/or degenerative disorders, inflammatory and/or degenerative conditions, or other inflammatory and/or degenerative processes. Suitable non-human animal subjects include canine, feline, equine, bovine, porcine, and the like. Illustratively, the subject may be a dog, a cat, a horse, a cow, a pig, other pets, other domestic livestock animals, and zoo animals, such as elephants, zebras, bears, pandas, kangaroos, monkeys, gorillas, baboons, other non-human primates, and the like. The subject can be one who has been diagnosed as suffering from an inflammatory and/or degenerative process, or the subject can be one who is susceptible to developing but who has not yet developed the inflammatory and/or degenerative process. Illustratively, the subject can be one who suffers from (or is susceptible to developing) one or more inflammatory and/or degenerative joint processes (e.g., osteoarthritis and/or rheumatoid arthritis). As further illustration, the subject can be one who suffers from (or is susceptible to developing) one or more inflammatory processes, such as chronic or other inflammatory processes of lung tissue, skin tissue, bowel tissue, or lamellar tissues (e.g., IAD, COPD, and other reactive airway diseases and processes; auto-immune dermatitis; chronic contact dermatitis (allergic or environmental); chronic laminitis; and inflammatory bowel disease). As yet further illustration, the subject can be one who suffers from (or is susceptible to developing) one or more degenerative processes (or is susceptible to developing) degenerative processes (such as Alzheimer's disease, atherosclerosis and arteriosclerosis, osteoarthritis and other degenerative joint diseases, Huntington's chorea, Parkinson's disease, optic atrophy, retinitis pigmentosa, macular degeneration, muscular dystrophy, and degenerative processes associated with aging). As still further illustration, the subject can be one who suffers from (or is susceptible to developing) one or more inflammatory processes (such as chronic or other inflammatory processes of lung tissue, skin tissue, bowel tissue, or lamellar tissues, examples of which include IAD, COPD, and other reactive airway diseases and processes, auto-immune dermatitis, chronic contact dermatitis (allergic or environmental), chronic laminitis, and inflammatory bowel disease) and also suffers from (or is susceptible to developing) degenerative processes (such as Alzheimer's disease, atherosclerosis and arteriosclerosis, osteoarthritis and other degenerative joint diseases, Huntington's chorea, Parkinson's disease, optic atrophy, retinitis pigmentosa, macular degeneration, muscular dystrophy, and degenerative processes associated with aging). As yet further illustration, the subject can be one who suffers from (or is susceptible to developing) one or more inflammatory processes (such as chronic or other inflammatory processes of lung tissue, skin tissue, bowel tissue, or lamellar tissues, examples of which include IAD, COPD, and other reactive airway diseases and processes, auto-immune dermatitis, chronic contact dermatitis (allergic or environmental), chronic laminitis, and inflammatory bowel disease) but who does not suffer from (and/or is not susceptible to developing) degenerative processes (such as Alzheimer's disease, atherosclerosis and arteriosclerosis, osteoarthritis and other degenerative joint diseases, Huntington's chorea, Parkinson's disease, optic atrophy, retinitis pigmentosa, macular degeneration, muscular dystrophy, and degenerative processes associated with aging). As still further illustration, the subject can be one who suffers from (or is susceptible to developing) one or more degenerative processes (such as Alzheimer's disease, atherosclerosis and arteriosclerosis, osteoarthritis and other degenerative joint diseases, Huntington's chorea, Parkinson's disease, optic atrophy, retinitis pigmentosa, macular degeneration, muscular dystrophy, and degenerative processes associated with aging) but who does not suffer from (and/or is not susceptible to developing) inflammatory processes (such as chronic or other inflammatory processes of lung tissue, skin tissue, bowel tissue, or lamellar tissues, examples of which include IAD, COPD, and other reactive airway diseases and processes, auto-immune dermatitis, chronic contact dermatitis (allergic or environmental), chronic laminitis, and inflammatory bowel disease).

As used herein, “inflammatory and/or degenerative processes” are meant to include inflammatory and/or degenerative diseases, inflammatory and/or degenerative disorders, and inflammatory and/or degenerative conditions. The phrase “inflammatory and/or degenerative” as used herein to modify diseases, disorders, conditions, and other processes, is meant to refer to diseases, disorders, conditions, and other processes which involve inflammation (e.g., chronic inflammation) and/or which involve degradation (e.g., chronic degradation), for example, of a subject's structural tissues or other tissues.

Degenerative processes are meant to refer to conditions in which there is a progressive impairment of both structure and function of a tissue or other part of the body excluding diseases caused by infection, inflammation, altered immune response, chemical or physical damage, or malignant change. Degenerative processes can be a normal part of aging, or they can be degenerative disorders. Generally, degenerative disorders are degenerative processes that begin earlier than degenerative processes associated with normal aging, that have a more rapid onset than degenerative processes associated with normal aging, that have a more rapid progression than degenerative processes associated with normal aging, and/or that affect some organs and not others. The degenerative disorder can be a chronic degenerative disorder, which implies a continuing disease process with progressive deterioration, often despite treatment. Examples of degenerative processes include Alzheimer's disease, atherosclerosis and arteriosclerosis, osteoarthritis and other degenerative joint diseases, Huntington's chorea, Parkinson's disease, optic atrophy, retinitis pigmentosa, macular degeneration, muscular dystrophy, and degenerative processes associated with aging.

Inflammatory processes are meant to include asthma (e.g., bronchial asthma, allergic aveolitis, etc.), dermatitis (e.g., atopic dermatitis, auto-immune dermatitis, allergic chronic contact dermatitis, environmental chronic contact dermatitis, and all other types of dermatitis except aging changes), laminitis (e.g., chronic laminitis), pemphigoid (e.g., Bullous pemphigoid), pemphigus, reactive airway disease (e.g., equine reactive airway disease, chronic obstructive pulmonary disease (“COPD”), inflammatory airway disease (“IAD”), recurrent airway obstruction (heaves), summer pasture associated obstructive pulmonary disease, etc.), inflammatory bowel disease (e.g., Crohn's disease, ulcerative colitis, etc.), multiple sclerosis (e.g., immune mediated multiple sclerosis, environmental multiple sclerosis, etc.), rheumatoid arthritis (e.g., autoimmune rheumatoid arthritis), periodontal disease, systemic lupus erythematosus, sarcoidosis, psoriasis, type I diabetes, and ischemia-reperfusion injury.

Illustratively, certain embodiments of the present invention are directed to the treatment of degenerative processes associated with particular body components, such as degenerative processes of lung tissue, skin tissue, bowel tissue, lamellar tissues, nerve tissue, connective tissue, vascular tissue, muscle tissue, skeletal tissue, blood components, an extracellular matrix, glands (e.g., spleen, thymus, endocrine glands, etc.), organs (e.g. liver, kidneys, etc.), and systems (e.g., endocrine system, immunologic system, etc.).

As further illustration, certain embodiments of the present invention are directed to the treatment of inflammatory processes associated with particular body components, such as inflammatory processes of lung tissue, skin tissue, bowel tissue, lamellar tissues, nerve tissue, connective tissue, vascular tissue, muscle tissue, skeletal tissue, blood components, an extracellular matrix, glands (e.g., spleen, thymus, endocrine glands, etc.), organs (e.g. liver, kidneys, etc.), and systems (e.g., endocrine system, immunologic system, etc.).

As further illustration, certain embodiments of the present invention are directed to the treatment of degenerative processes associated with aging, inflammatory and/or degenerative processes resulting from infectious agents, inflammatory and/or degenerative processes resulting from physical insult (e.g., trauma, radiation, cold, heat, etc.), inflammatory and/or degenerative processes resulting from tumorogenesis and/or metastasis, inflammatory and/or degenerative processes resulting from chemical insult (e.g., drugs, toxins, alcohol, etc.), inflammatory and/or degenerative processes resulting from oxidative stress, and/or inflammatory and/or degenerative processes that are immune mediated.

As still further illustration, certain embodiments of the present invention are directed to the treatment of inflammatory and/or degenerative process selected from the group consisting of chronic inflammatory disease, geriatric wasting, cancer cachexia, cachexia associated with chronic inflammation, sick feeling syndrome (which is meant to refer to any diseases, disorders and other syndromes resulting from adverse effects of TNFα on the central nervous system), and combinations thereof.

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named inflammatory and/or degenerative disease, inflammatory and/or degenerative disorder, inflammatory and/or degenerative condition, or other inflammatory and/or degenerative process. As such, the treatment methods of this invention encompass both therapeutic and prophylactic administration.

The treatment methods of the present invention are practiced by administering a composition of the present invention to the subject. Typically, the compositions are administered orally in an effective amount. As used herein, the term “effective amount” refers to the amount or dose of the composition, upon single or multiple dose administration to the subject, which provides the desired effect in the subject under diagnosis or treatment.

An effective amount can be readily determined by the attending diagnostician (or others skilled in the art) by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the inflammatory and/or degenerative disorder, disease, or condition involved; the response of the individual subject; the composition's formulation; the mode of administration; the bioavailability characteristics of the composition administered; the dose regimen selected; the use of concomitant medication; and other relevant circumstances.

A typical daily dose can contain from about 0.01 mg/kg to about 500 mg/kg (such as from about 0.05 mg/kg to about 200 mg/kg and/or from about 0.1 mg/kg to about 25 mg/kg) of active ingredients (e.g., the curcuminoid, polymethoxylated flavone, catechin, and boswellic acid components and the optional extracts) in the aggregate. The composition can be administered in any suitable form (e.g., pills, elixir, powders, etc.), and it can be administered directly or it can be mixed with or otherwise incorporated into the subject's food or drink in amounts such that the desired daily dose is achieved.

In one illustrative embodiment, the method of the present invention can be practiced using compositions that are formulated in a unit dosage form, each dosage containing from about 1 mg to about 2 g (e.g., from about 2 mg to about 1 g, and/or from about 5 mg to about 500 mg) of the curcuminoid, polymethoxylated flavone, catechin, and boswellic acid components. The term “unit dosage form” refers to a physically discrete unit suitable as unitary dosages for a subject, each unit containing a predetermined quantity of active ingredients calculated to produce the desired therapeutic effect, in association with a suitable carriers, diluents, or excipients.

As one skilled in the art will appreciate, the formulation can be prepared with materials (e.g., actives excipients, carriers, diluents, etc.) having properties (e.g., purity) that render the formulation suitable for administration to humans. Alternatively, the formulation can be prepared with materials having purity and/or other properties that render the formulation suitable for administration to non-human subjects but not suitable for administration to humans.

As one skilled in the art will also appreciate, the composition described herein can be formulated so as to carry a minimum of adverse side effects and result in similar or improved efficacy in the management of the aforementioned inflammatory and degenerative conditions relative to conventional therapies (e.g., those involving the administration of NSAIDS, glucocorticoids, and/or immunosuppressive agents). Moreover, inhibition of the degenerative process (as opposed to a mere treatment of the symptoms) would be a desirable (but not a necessary) component of the therapies described herein. The compositions described herein can be suitable for long term use alone; useful as an adjunct therapy along with NSAIDS, glucocorticoids, or immunosuppressive agents; and/or useful in a program involving rotation between any or all of these agents, thereby decreasing long term exposure to (and, therefore, side effects resulting from) any one agent.

In further aspects thereof, the present invention also relates to compositions for treating an inflammatory and/or degenerative process in a human or other animal in which the composition includes a curcuminoid, a catechin, and a boswellic acid. Such compositions can also include additional components, such as one or more of the following: a Harapagophytum procumbens extract, a Trypterigium wilfordii Hook extract, a Glycyrrhiza glabra extract, a Cinnamomum cassia extract, a Magnolia obovata extract, a Magnolia officianalis extract, and a Euonymus alatus extract.

In still further aspects thereof, the present invention also relates to methods for treating an inflammatory and/or degenerative process in a subject by inhibiting ADAM17 activity in the subject, for example, by using one of the compositions of the present invention.

In yet further aspects thereof, the present invention also relates to methods for treating an inflammatory and/or degenerative process in a subject by up-regulating TIMP3 activity in the subject, for example, by using one of the compositions of the present invention.

The present invention is also illustrated by the following non-limitative examples.

### EXAMPLES

### Example 1

### Studies into the Effects of Formulations of the Present Invention

Matrix metalloproteinase (“MMP”) inhibitors intervene in the inflammatory process by virtue of their ability to limit the MMP activation of the chemokines responsible for macrophage, monocyte and neutrophil attraction to tissue. Inhibition of MMPs also results in decreased enzymatic tissue destruction, particularly that of cartilage.

Our formula, which contains natural MMP inhibitors, was designed to offer an alternative to currently available therapies for protection of joints in equine athletes. Current therapy includes NSAIDs, PSGAGs, hyaluronate derivatives, nutraceuticals, and intra-articular injections. These therapies are intended to mitigate an existing inflammatory state or, in the case of nutraceuticals, to provide the nutrients required for the rebuilding of degraded cartilage.

We believe that our blend of MMP inhibitors represents a preferred method for the prevention of joint damage due to athletic trauma. It is believed that by inhibiting inflammatory cell infiltration and cartilage degradation, joint injury can be avoided, rather than treated after the fact. Our formula provides results on a par with other more expensive forms of therapy at a cost similar to that of nutraceuticals. In addition, our formula does not come with the negative side effects associated with NSAIDS and corticosteroids, nor the risks associated with intra-articular injection.

Clinical studies were conducted on equine orthopedic cases using one of the following two formulations. The clinical studies initially involved about 50 such cases but have now been expanded to include over 200 horses.

Formulation A provided 1 g of tetrahydrocurcumin (98% extract), 2 g of Boswellia seratta (65% extract), and 1.25 g of Glycyrrhiza glabra (20% extract).

Formulation B provided 1 g of tetrahydrocurcumin (98% extract), 2 g of Boswellia seratta (65% extract), 0.5 g of polymethoxylated flavone (from Citrus reticulata peel), and 0.75 g of Glycyrrhiza glabra (20% extract).

Formulation A was used, for example, in jurisdictions with drug testing because the polymethoxylated flavone (from Citrus reticulata peel) of Formulation B may contain a trace quantity of synephrine. In cases where drug testing is not an issue (e.g., as in cases involving pleasure and geriatric horses), Formulation B was employed.

The appropriate formulation (i.e., either Formulation A or Formulation B) was administered orally twice daily to effect, or, in the case of incurable chronic inflammatory and degenerative disorders, for as long as the conditions exacerbating the disorder existed.

As part of our clinical studies on equine orthopedic cases, we made the following observations: (1) that the results were equal to or better than those obtained with NSAIDS; (2) that the formula was effective even in some cases where intra-articular injections no longer provided a satisfactory outcome; (3) that the formula was effective even in some cases where all other therapies had failed and euthanasia was recommended; (4) that the formula was effective in a few cases of non-inflammatory diseases, such as navicular disease and stringhalt; and (5) that concurrent, non-orthopedic conditions were unexpectedly affected.

With regard to observation (5), we noted that chronic unresponsive dermatitis resolved; that chronic inflammatory airway disease resolved; that chronic inflammatory bowel disease resolved; and that rejuvenation of geriatrics occurred.

With regard to the unexpected results in observation (4) above, these results were reproducible. Navicular disease, an avascular necrosis of the navicular bone of the horse, is a common cause of unsoundness and impaired performance in show and sport horses. Current therapy involves NSAIDS, intra-articular injections, vasodilating agents, and surgical denervation of the affected area. Our formula provides a safe, effective alternative to these therapies, free from the adverse effects associated with them. Stringhalt is an uncontrollable muscle spasm of the lateral digital extensor tendon of the hind limb of horses. The etiology of stringhalt is unknown. The only treatment option is surgical resection of the lateral digital extensor tendon. Although this condition is rare, we were able to reproduce positive results in 3/3 cases.

The unexpected results experienced in non-orthopedic conditions as previously described in observation (5), above, led us to expand our trials to clinically related cases and to include canine and human patients.

The human trials initially involved 2 subjects but have now been expanded to over 50 subjects. The human subjects were administered a formulation made by combining 75 g of tetrahydrocurcumin (98% extract), 100 g of Boswellia seratta (65% extract), 37.5 g of Citrus reticulata peel (5:1 extract), and 37.5 g of Camellia sinensis (80% catechin extract). The formulation was packaged in single 0 (“0”) capsules, with each capsule containing 75 mg of tetrahydrocurcumin (98% extract), 100 mg of Boswellia seratta (65% extract), 37.5 mg of Citrus reticulata peel (5:1 extract), and 37.5 mg of Camellia sinensis (80% catechin extract), the balance of the 0 capsules being rice flour and magnesium stearate extenders. The capsules were administered to the human subjects (100-200 lbs body weight) as needed and provided 0.375 mg to 0.75 mg of tetrahydrocurcumin (98% extract) per lb of subject body weight, 0.5 mg to 1 mg of Boswellia seratta (65% extract) per lb of subject body weight, 0.1875 mg to 0.375 mg of Citrus reticulata peel (5:1 extract) per lb of subject body weight, and 0.1875 mg to 0.375 mg of Camellia sinensis (80% catechin extract) per lb of subject body weight.

The canine trials initially involved 6 subjects but have now been expanded to over 40 subjects. The canine subjects were administered a formulation made by combining 75 g of tetrahydrocurcumin (98% extract), 100 g of Boswellia seratta (65% extract), 37.5 g of Citrus reticulata peel (5:1 extract), and 37.5 g of Camellia sinensis (80% catechin extract). The formulation was packaged in double 0 (“00”) capsules, with each capsule containing 37.5 mg of tetrahydrocurcumin (98% extract), 50 mg of Boswellia seratta (65% extract), 18.75 mg of Citrus reticulata peel (5:1 extract), and 18.75 mg of Camellia sinensis (80% catechin extract), the balance of the 00 capsules being extenders. The capsules were administered to the canine subjects (50-100 lbs body weight) as needed and provided 0.375 mg to 0.75 mg of tetrahydrocurcumin (98% extract) per lb of subject body weight, 0.5 mg to 1 mg of Boswellia seratta (65% extract) per lb of subject body weight, 0.1875 mg to 0.375 mg of Citrus reticulata peel (5:1 extract) per lb of subject body weight, and 0.1875 mg to 0.375 mg of Camellia sinensis (80% catechin extract) per lb of subject body weight.

Positive results were obtained in cases of pemphigus, severe unresponsive pruritis, Crohns disease, inflammatory bowel disease, asthma, inflammatory airway disease, allergic rhinitis, allergic bronchitis, COPD and geriatric wasting (inappetance, depression, catabolic state). Many of these cases showed a dramatic response to our formula, which could not be explained by MMP inhibitory activity alone. These chronic inflammatory diseases (“CIDs”) involve different systems, but, at their core, is an unregulated cycle of inflammation with elevated tumor necrosis factor alpha (“TNFα”) levels as a component. This suggested that the formula's effectiveness against CIDs may be attributable to its ability to inhibit TNFα which has a central role in perpetuating the cycle of inflammation.

A fundamental question arose: are these diseases of excessive TNFα due to unrelenting stimulation and up-regulation by exogenous factors, or are they diseases of dysregulation, involving the system that normally regulates TNFα and the cycle of inflammation? Since organisms in a similar environment are subjected to equal exposure to exogenous factors, the regulatory mechanisms must differ between the CID suffer and non-sufferer. TNFα is up-regulated by any bacteria or microbe, many cytokines, Tcell surface molecules, ischemia, trauma, radiation, oxidative stress, and UV light. It is a constitutive cytokine needed for initial response to pathogens, acute phase response/innate immune response, Th1/acquired immunity, wound healing, tumor surveillance, and regulation of energy metabolism. When TNFα is up-regulated, it simultaneously initiates expression of factors (TIMPs, sTNFr, Interleukin 10) that would limit the inflammatory response's intensity and duration, resulting in an appropriate self-limiting response to the initiating factor.

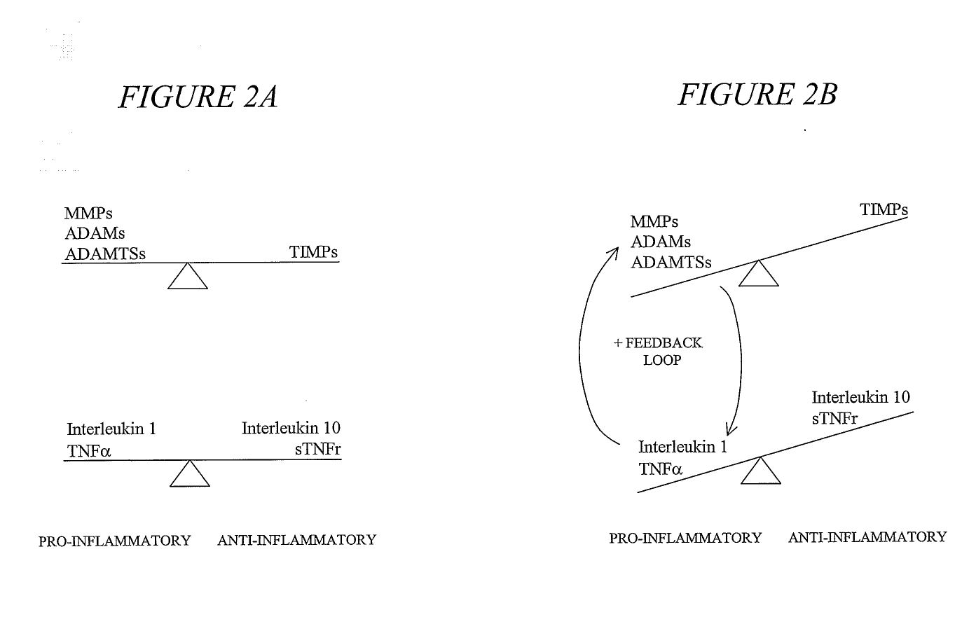

Under normal conditions, TNFα up-regulates both MMPs and TIMPs (tissue inhibitor of metalloproteinases) in an attempt to maintain a 1:1 ratio resulting in stoichiometric inhibition. Since TNFα up-regulates the MMP/TIMP axis in an attempt to self-regulate, we propose that in these chronic inflammatory diseases, a balanced up-regulation is not achieved, leading to over-expression of TNFα, MMPs, ADAMs (a disintegrin and metalloproteinase) and ADAMTSs (a disintegrin and metalloproteinase with thrombospondin motif) and under-expression of TIMPs and other control mechanisms. This is illustrated in FIG. 2. The imbalance is magnified by a positive feedback loop between TNFα and IL1, and the metalloproteinases, ADAMs, and ADAMTSs. The result is the chronic inflammatory cycle present in CIDs.

Current cutting edge therapy of these CIDs involves treating the symptom-elevated TNFα. This can be achieved by: (1) inhibiting transcription of TNFα (corticosteroids); (2) binding TNFα (soluble receptors); and (3) binding TNFα (anti-TNFα antibodies). All of these therapies have in common the negative side effect of allowing opportunistic infection due to blockage of TNFα and the beneficial role it plays in host defense. Some of these therapies are expensive and require repeated injections. The disadvantages are many, and they only provide symptomatic relief, often short-lived symptomatic relief.

In certain embodiments of the present invention, our formulations contain a broad spectrum of MMP inhibitors. MMPs 1, 2, 3, 7, 9, 12, and 14 have been reported to be very weak converters of pro-TNFα to TNFα. Over 90% of TNFα activation occurs via ADAM17. It has become evident that, in order to achieve the degree of clinical response we have observed, our formula must also inhibit ADAM17 in addition to the MMPs previously described. Components of our formula are known to up-regulate TIMP 1 and 2. These TIMPs are considered to have little if any ADAM17 regulatory (inhibitory) activity. We propose that our formulations may also stimulate TIMP3 up-regulation, as TIMP3 is an effective ADAM17 inhibitor. This formulation, therefore, represents the only known natural ADAM17 inhibitor and TIMP3 promoter, thus explaining its powerful TNFα inhibitory activity, and it's unexpected efficacy in the aforementioned CIDS. ADAM17 is required for the activation of TNFα to its active form. TIMP3 is the only known biochemical inhibitor of ADAM17. ADAM 17 is over-expressed and TIMP3 is under-expressed in tissue involved in CIDs. Restoring TIMP3 to normal levels of expression in these locally deficient tissues will, it is believed, rebalance the regulatory axis of MMPS/TIMPS and cytokines and disrupt the chronic cycle of inflammation without interfering with the TNFα induced inflammatory and immune response to challenges faced by the biological system as a whole.

While not intending to be limited to any theory of operation, it is believed that rebalancing of the natural homeostatic mechanisms responsible for maintaining an appropriate inflammatory response has been achieved with the clinical application of our formula; and the MMP/TIMP/cytokine axis is restored to normal. During clinical use, our patients experienced satisfactory to excellent resolution of the conditions undergoing treatment by our formula. If challenged by a pathogen, toxin, or trauma during the course of treatment (including long term treatment) acute phase response, innate immune response, and acquired immune response were uninhibited in their activity. Clinically normal immune and inflammatory responses occurred in a self-limiting, appropriate fashion concurrent with the treatment of CID. This confirms that our formula is not immunosuppressive or anti-TNFα or anti-cytokine in nature. Again, while not intending to be limited by any theory of operation, it is believed that restoration of the normal balanced, symptom-free state is achieved by encouraging a state of immune and inflammatory self-regulation. We know of no other formula, natural or synthetic, that can rebalance the MMP/TIMP/cytokine axis. Additional evidence for the restoration of this self-regulating state comes from our patients' experiences. After an induction phase, many patients reduce dosage and/or frequency of dosage, or they find they can discontinue treatment until faced with an extraordinary challenge, indicating that the self-regulatory state can endure beyond the end of the treatment regimen.

Our human patients have reported a more positive attitude, more energy and general feeling of well being. At first we attributed this to the relief of symptoms and the optimism associated with less dependency on medication and a shift to self-regulation. Our animal population was observed to demonstrate similar behaviors (although not verbalized). We discount psychological or placebo effect as a factor in this population, and tend to view our human patients' reports with more credibility with this in mind. TNFα has been implicated as a cause of “sick feeling” behaviors resulting from its activity within the central nervous system (“CNS”). Current anti-TNFα therapy involves the use of large macro-molecules that are not able to enter the CNS and brain. Our formula's influence on this symptom of CID indicates that biologically active components may cross the blood-brain barrier. We know of no other method to accomplish the mitigation of CNS induced symptoms of chronic illness.

These positive changes were especially noticeable in geriatric patients. Our geriatric patients have “broken the cycle” of CID/degeneration/catabolism and have returned to an anabolic state that has been maintained after discontinuation of treatment. Inappetance, weight loss, malaise, listlessness, fatigue, and depression have consistently been replaced by improved appetite, weight gain, increased activity level, and a restoration of the will to live (playful, engaged, animated behavior). We have, on numerous occasions, observed clinical rejuvenation of animals literally on the verge of euthanasia. The potential use for support of geriatric and cancer patients seems clear, to achieve both an improvement in attitude and a return to the anabolic state.

In summary, we have developed a plant based formula that appears to demonstrate MMP inhibitory activity and TNFα inhibitory activity. This inhibition is unlike that obtained with any other cytokine inhibitory based strategy. Clinical experience indicates that the formula targets only the inappropriate excessive and prolonged TNFα/MMP elevations associated with CIDs. Immune and inflammatory responses of an appropriate, self-limiting nature are not suppressed. Currently available suppressive therapies cannot achieve this degree of specificity. These desirable characteristics can only be explained by the formula's encouraging restoration of the deficient self-regulatory mechanism of TIMP3 over ADAM17 over TNFα activation. Restoration of the balance between inflammatory and anti-inflammatory mediators also permits the disease free state to endure beyond the termination of treatment.

Excess TNFα plays a central role in a wide variety of inflammatory disorders (e.g., asthma, atherosclerosis, Crohn's disease, congestive heart failure, chronic obstructive pulmonary disease, geriatric wasting, inflammatory bowel disease and associated osteoporosis, insulin resistance, neurodegenerative diseases, osteoarthritis, pemphigus, psoriasis, psoriatic arthritis, rheumatoid arthritis, and spondylitis). Current therapy of these disorders is not curative, has limited efficacy and many side effects. Our formulation provides a safe, effective and inexpensive alternative to management of these conditions, possibly due to our formula's unique ability to re-establish homeostasis of the MMP/TIMP/cytokine axis.

### Example 2

### Illustrative Formulations

The following tables provide an illustrative list of formulations suitable for use in the treatment methods and compositions of the present invention. The following is provided only to illustrate the invention and should not be interpreted as limiting the present invention in any way.

### Formulation 1

The following formulation inhibits MMP 3, MMP 9, ADAMTS-4, and MMP 13 by interfering with MMP 3, MMP 9, ADAMTS-4, and MMP 13 production. It contains 60 g of tetrahydrocurcumin (98% extract), 120 g of Boswellia seratta (65% extract), and 75 g of Glycyrrhiza glabra (20% extract) (for palatability). It is administered so as to deliver 1-2 mg of tetrahydrocurcumin (98% extract) per pound and so as to deliver 2-4 mg of Boswellia seratta (65% extract) per pound.

### Formulation 1A

The following formulation inhibits MMP 3, MMP 9, ADAMTS-4, and MMP 13 by interfering with MMP 3, MMP 9, ADAMTS-4, and MMP 13 production. It contains 60 g of tetrahydrocurcumin (98% extract), 120 g of Boswellia seratta (65% extract), 30 g of Citrus reticulata peel (5:1 extract), and 45 g of Glycyrrhiza glabra (20% extract) (for palatability). It is administered so as to deliver 1-2 mg of tetrahydrocurcumin (98% extract) per pound and so as to deliver 2-4 mg of Boswellia seratta (65% extract) per pound.

### Formulation 2

The following formulation inhibits MMP 1, MMP 3, MMP 9, ADAMTS-4, and MMP 13 by interfering with MMP 1, MMP 3, MMP 9, ADAMTS-4, and MMP 13 production. It contains 60 g of tetrahydrocurcumin (98% extract), 60 g of Boswellia seratta (65% extract), 100 g of Citrus reticulata peel (5:1 extract), and 35 g of Glycyrrhiza glabra (20% extract) (for palatability). It is administered so as to deliver 1-2 mg of tetrahydrocurcumin (98% extract) per pound, so as to deliver 1-2 mg of Boswellia seratta (65% extract) per pound, and so as to deliver 1.6-3.3 mg of Citrus reticulata peel (5:1 extract) per pound.

### Formulation 3

The following formulation inhibits MMP 1, MMP 3, MMP 9, ADAMTS-4, and MMP 13 by interfering with MMP 1, MMP 3, MMP 9, ADAMTS-4, and MMP 13 production. It contains 45 g of tetrahydrocurcumin (98% extract), 60 g of Boswellia seratta (65% extract), 50 g of Citrus reticulata peel (5:1 extract), 50 g of Cinnamomum cassia (5:1 extract), 50 g of Magnolia officianalis (5:1 extract), and 30 g of Glycyrrhiza glabra (20% extract) (for palatability). It is administered so as to deliver 0.75-1.5 mg of tetrahydrocurcumin (98% extract) per pound, so as to deliver 1-2 mg of Boswellia seratta (65% extract) per pound, and so as to deliver 0.8-1.7 mg of each of Citrus reticulata peel (5:1 extract), Cinnamomum cassia (5:1 extract), and Magnolia officianalis (5:1 extract) per pound.

### Formulation 4

The following formulation inhibits MMP 1, MMP 2, MMP 3, MMP 7, MMP 9, ADAMTS-4, MMP 13, and MMP 14 by interfering with MMP 1, MMP 2, MMP 3, MMP 7, MMP 9, ADAMTS-4, MMP 13, and MMP 14 production. It contains 45 g of tetrahydrocurcumin (98% extract), 60 g of Boswellia seratta (65% extract), 60 g of Citrus reticulata peel (5:1 extract), 30 g of Cinnamomum cassia (5:1 extract), 30 g of Magnolia officianalis (5:1 extract), 30 g of Glycyrrhiza glabra (20% extract) (for palatability), and 30 g of Camellia sinensis (80% catechin extract). It is administered so as to deliver 0.75-1.5 mg of tetrahydrocurcumin (98% extract) per pound, so as to deliver 0.5-1 mg of Camellia sinensis (80% catechin extract) per pound, so as to deliver 1-2 mg of each of Boswellia seratta (65% extract) and Citrus reticulata peel (5:1 extract) per pound, and so as to deliver 0.5-1 mg of each of Cinnamomum cassia (5:1 extract) and Magnolia officianalis (5:1 extract) per pound.

### Formulation 5

Hard gelatin capsules can be prepared using the following ingredients:

| | Quantity |
|---|---|
| | (mg/capsule) |
| Curcuminoid | 100 |
| Polymethoxylated flavone | 50 |
| Catechin | 50 |
| Boswellic acid | 50 |
| Starch, dried | 200 |
| Magnesium stearate | 10 |
| Total | 460 mg |

The above ingredients are mixed and filled into hard gelatin capsules in 460 mg quantities.

### Formulation 6

A tablet in accordance with the present invention can be prepared using the ingredients below:

| | Quantity |
|---|---|
| | (mg/tablet) |
| Curcuminoid | 80 |
| Polymethoxylated flavone | 80 |
| Catechin | 45 |
| Boswellic acid | 45 |
| Cellulose, microcrystalline | 400 |
| Silicon dioxide, fumed | 10 |
| Stearic acid | 5 |
| Total | 665 mg |

The components are blended and compressed to form tablets each weighing 665 mg.

### Formulation 7

Tablets each containing 60 mg of total active ingredient can be made as follows:

| | | |
|---|---|---|
| Curcuminoid | 10 | mg |
| Polymethoxylated flavone | 20 | mg |
| Catechin | 10 | mg |
| Boswellic acid | 20 | mg |
| Starch | 45 | mg |
| Microcrystalline cellulose | 35 | mg |
| Polyvinylpyrrolidone | 4 | mg |
| Sodium carboxymethyl starch | 4.5 | mg |
| Magnesium stearate | 0.5 | mg |
| Talc | 1 | mg |
| Total | 150 | mg |

The active ingredients, starch, and cellulose are passed through a No. 45 mesh U.S. sieve and mixed thoroughly. The solution of polyvinylpyrrolidone is mixed with the resultant powders which are then passed through a No. 14 mesh U.S. sieve. The granules so produced are dried at 50° C. and passed through a No. 18 mesh U.S. sieve. The sodium carboxymethyl starch, magnesium stearate, and talc, previously passed through a No. 60 mesh U.S. sieve, are then added to the granules which, after mixing, are compressed on a tablet machine to yield tablets each weighing 150 mg.

### Formulation 8

Capsules each containing 80 mg of total active ingredient can be made as follows:

| | | |
|---|---|---|
| Curcuminoid | 15 | mg |
| Polymethoxylated flavone | 15 | mg |
| Catechin | 15 | mg |
| Boswellic acid | 15 | mg |
| Trypterigium wilfordii Hook extract | 10 | mg |
| Magnolia obovata extract | 10 | mg |
| Starch | 59 | mg |
| Microcrystalline cellulose | 59 | mg |
| Magnesium stearate | 2 | mg |
| Total | 200 | mg |

The active ingredients, cellulose, starch, and magnesium stearate are blended, passed through a No. 45 sieve, and filled into hard gelatin capsules in 200 mg quantities.

### Formulation 9

Suspensions each containing 50 mg of total active ingredient per 5 ml dose can be made as follows:

| Curcuminoid | 10 | mg |
|---|---|---|
| Polymethoxylated flavone | 10 | mg |
| Catechin | 10 | mg |
| Boswellic acid | 10 | mg |
| Trypterigium wilfordii Hook extract | 5 | mg |
| Harapagophytum procumbens extract | 5 | mg |
| Sodium carboxymethyl cellulose | 50 | mg |
| Syrup | 1.25 | ml |
| Benzoic acid solution | 0.10 | ml |
| Flavor | q.v. | |
| Color | q.v. | |
| Purified water to total | 5 | ml |

The active ingredients are passed through a No. 45 mesh U.S. sieve and mixed with the sodium carboxymethyl cellulose and syrup to form a smooth paste. The benzoic acid solution, flavor, and color are diluted with some of the water and added with stirring. Sufficient water is then added to produce the required volume.

### Formulation 10

A dry cat food preparation containing 2.3 g of total active ingredient per 500 g portion can be prepared as follows:

| | |
|---|---|
| Curcuminoid | 400 mg |
| Polymethoxylated flavone | 400 mg |
| Catechin | 400 mg |
| Boswellic acid | 400 mg |
| Harapagophytum procumbens extract | 100 mg |
| Trypterigium wilfordii Hook extract | 100 mg |
| Glycyrrhiza glabra extract | 100 mg |
| Cinnamomum cassia extract | 100 mg |
| Magnolia obovata extract | 100 mg |
| Magnolia officianalis extract | 100 mg |
| Euonymus alatus extract | 100 mg |
| Dry cat food mixture | 500 g |

The dry cat food mixture can contain ground yellow corn, corn gluten meal, soybean meal, poultry by-product meal, animal fat, fish meal, meat and bone meal, ground wheat, phosphoric acid calcium carbonate, dried animal digest, salt, brewers dried yeast, potassium chloride, dried whey solubles, choline chloride, dried skimmed milk, taurine, L-lysine, zinc oxide, ferrous sulfate, niacin, vitamin A, vitamin D₃, vitamin B₁₂, calcium pantothenate, citric acid, manganese sulfate, riboflavin supplement, biotin, copper salt, thiamine mononitrate, pyridoxine hydrochloride, menadione sodium bisulfate complex, such that the crude protein is not less than 31%, crude fat is not less than 8%, crude fiber is not more than 4.5%, moisture is not more than 12%, calcium is not less than 1.2%, phosphorous is not less than 1.0%, sodium chloride is not more than 1.5%, the metabolizable energy is about 3,600 kcal/kg, taurine, iron, vitamins A, D₃, B₁₂, and E are at least 100% of levels recommended by the Association of American Feed Control Officials. The active ingredients are combined and passed through a No. 45 mesh U.S. sieve and then tumbled with the dry cat food mixture to produce the dry cat food preparation containing 2.3 g of total active ingredient per 500 g portion.

### Formulation 11

A multivitamin and mineral dietary supplement in tablet form containing 500 mg of total active ingredient per tablet suitable for older human adults can be prepared so at to contain the following: curcuminoid (100 mg), polymethoxylated flavone (50 mg), catechin (50 mg), boswellic acid (50 mg), Harapagophytum procumbens extract (50 mg), Trypterigium wilfordii Hook extract (50 mg), Glycyrrhiza glabra extract (50 mg), Cinnamomum cassia extract (50 mg), Magnolia obovata extract (25 mg), Magnolia officianalis extract (25 mg), calcium carbonate, calcium phosphate (200 mg Ca, 20% RDI; 48 mg phosphorous, 5% RDI), magnesium oxide, magnesium stearate (100 mg, 25% RDI), potassium chloride (80 mg, 2% RDI), microrystalline cellulose, ascorbic acid (60 mg, 100% RDI), gelatin, d l-alpha-tocopherylacetate (45 I.U., 150% RDI), modified food starch, maltodextrin, crospovidone, reduced iron (4 mg, 22 RDI), hydroxypropyl methylcellulose, niacinamide (20 mg, 100% RDI), zinc oxide (15 mg, 100% RDI), calcium pantothenate, manganese sulfate (3.5 mg), vitamin D (400 I.U., 100% RDI), titanium dioxide, vitamin A and β-carotene (5000 I.U., 100% RDI), stearic acid, pyridoxine hydrochloride (3 mg, 150% RDI), riboflavin (1.7 mg, 100% RDI), silicon dioxide, copper oxide (2 mg, 100% RDI), dextrose, thiamin mononitrate (1.5 mg, 100% RDI), triethyl citrate, polysorbate 80, chromium chloride (130 μg), artificial colors, potassium iodide (150 μg, 100% RDI), sodium metasilicate (2 mg), sodium molybdate (160 μg), borates, sodium selenate (20 μg), biotin (30 μg, 10% RDI), sodium metavanadate (10 μg), cyanocobalamin (25 μg, 417% RDI), nickelous sulfate (5 μg), and phytonadione.

Although the invention has been described in detail for the purpose of illustration, it is understood that such detail is solely for that purpose, and variations can be made therein by those skilled in the art without departing from the spirit and scope of the invention which is defined by the claims that are set forth below.

What is claimed is:

1. A composition for treating an inflammatory and/or degenerative process in a human or other animal, said composition comprising at least four of the following: a MMP 1 inhibitor; a MMP 2 inhibitor; a MMP 3 inhibitor; a MMP 7 inhibitor; a MMP 9 inhibitor; an ADAMTS-4 inhibitor; a MMP 13 inhibitor; and a MMP 14 inhibitor.

2. A composition according to claim 1, wherein said composition comprises a MMP 1 inhibitor and wherein said MMP 1 inhibitor comprises a polymethoxylated flavone and a catechin.

3. A composition according to claim 1, wherein said composition comprises a MMP 2 inhibitor and wherein said MMP 2 inhibitor comprises a catechin.

4. A composition according to claim 1, wherein said composition comprises a MMP 3 inhibitor and wherein said MMP 3 inhibitor comprises a curcuminoid, a polymethoxylated flavone, and a boswellic acid.

5. A composition according to claim 1, wherein said composition comprises a MMP 7 inhibitor and wherein said MMP 7 inhibitor comprises a catechin.

6. A composition according to claim 1, wherein said composition comprises a MMP 9 inhibitor and wherein said MMP 9 inhibitor comprises a catechin, a polymethoxylated flavone, and a curcuminoid.

7. A composition according to claim 1, wherein said composition comprises an ADAMTS-4 inhibitor and wherein said ADAMTS-4 inhibitor comprises a boswellic acid and a curcuminoid.

8. A composition according to claim 1, wherein said composition comprises a MMP 13 inhibitor wherein said MMP 13 inhibitor comprises a catechin, a boswellic acid, and a curcuminoid.

9. A composition according to claim 1, wherein said composition comprises a MMP 14 inhibitor wherein said MMP 14 inhibitor comprises a catechin.

10. A composition according to claim 1, wherein said composition comprises at least 5 of the following: a MMP 1 inhibitor, a MMP 2 inhibitor, a MMP 3 inhibitor, a MMP 7 inhibitor, a MMP 9 inhibitor, an ADAMTS-4 inhibitor, a MMP 13 inhibitor, and a MMP 14 inhibitor.

11. A composition according to claim 1, wherein said composition comprises at least 6 of the following: a MMP 1 inhibitor, a MMP 2 inhibitor, a MMP 3 inhibitor, a MMP 7 inhibitor, a MMP 9 inhibitor, an ADAMTS-4 inhibitor, a MMP 13 inhibitor, and a MMP 14 inhibitor.

12. A composition according to claim 1, wherein said composition comprises at least 7 of the following: a MMP 1 inhibitor, a MMP 2 inhibitor, a MMP 3 inhibitor, a MMP 7 inhibitor, a MMP 9 inhibitor, an ADAMTS-4 inhibitor, a MMP 13 inhibitor, and a MMP 14 inhibitor.

13. A composition according to claim 1, wherein said composition comprises each of the following: a MMP 3 inhibitor, a MMP 9 inhibitor, an ADAMTS-4 inhibitor, and a MMP 13 inhibitor.

14. A composition according to claim 1, wherein said composition comprises each of the following: a MMP 1 inhibitor, a MMP 3 inhibitor, a MMP 9 inhibitor, an ADAMTS-4 inhibitor, and a MMP 13 inhibitor.

15. A composition according to claim 1, wherein said composition comprises each of the following: a MMP 1 inhibitor, a MMP 2 inhibitor, a MMP 3 inhibitor, a MMP 7 inhibitor, a MMP 9 inhibitor, an ADAMTS-4 inhibitor, a MMP 13 inhibitor, and a MMP 14 inhibitor.

16. A composition according to claim 15, wherein said MMP 1 inhibitor comprises a polymethoxylated flavone and a catechin; wherein said MMP 2 inhibitor comprises a catechin; wherein said MMP 3 inhibitor comprises a curcuminoid, a polymethoxylated flavone, and a boswellic acid; wherein said MMP 7 inhibitor comprises a catechin; wherein said MMP 9 inhibitor comprises a catechin, a polymethoxylated flavone, and a curcuminoid; wherein said ADAMTS-4 inhibitor comprises a boswellic acid and a curcuminoid; wherein said MMP 13 inhibitor comprises a catechin, a boswellic acid, and a curcuminoid; and wherein said MMP 14 inhibitor comprises a catechin.

17. A method for treating an inflammatory and/or degenerative process in a subject, said method comprising:
administering to the subject a composition according to claim 1.

18. A method according to claim 17, wherein the composition is administered to the subject in the form of a pill.

19. A method according to claim 17, wherein the composition is administered to the subject in the form of a powder or granules.

20. A method according to claim 17, wherein the composition is administered to the subject in the form of a liquid.

21. A method according to claim 17, wherein the composition is administered to the subject in the form of a food preparation.

22. A method according to claim 17, wherein the subject is a human.

23. A method according to claim 17, wherein the subject is a non-human animal.

24. A method according to claim 17, wherein the subject is selected from the group consisting of a horse, a dog, and a cat.

25. A method according to claim 17, wherein the subject is a zoo animal.

26. A method according to claim 17, wherein the inflammatory and/or degenerative process is selected from the group consisting of Alzheimer's disease, atherosclerosis or arteriosclerosis, degenerative joint diseases, Huntington's chorea, Parkinson's disease, optic atrophy, retinitis pigmentosa, macular degeneration, muscular dystrophy, asthma, dermatitis, laminitis, pemphigoid, pemphigus, reactive airway disease, inflammatory bowel disease, multiple sclerosis, rheumatoid arthritis, periodontal disease, systemic lupus erythematosus, sarcoidosis, psoriasis, type I diabetes, ischemia-reperfusion injury, and combinations thereof.

27. A method according to claim 17, wherein the inflammatory and/or degenerative process is an inflammatory and/or degenerative joint process.

28. A method according to claim 17, wherein the inflammatory and/or degenerative process is an inflammatory and/or degenerative joint process selected from the group consisting of osteoarthritis and rheumatoid arthritis.

29. A method according to claim 17, wherein the inflammatory and/or degenerative process is a result of infectious agents, is a result of physical insult, is a result of tumorogenesis and/or metastasis, is a result of chemical insult, is a result of oxidative stress, is immune mediated, and/or is a degenerative processes associated with aging.

30. A method according to claim 17, wherein the inflammatory and/or degenerative process is an inflammatory process of lung tissue, skin tissue, bowel tissue, lamellar tissue, nerve tissue, connective tissue, vascular tissue, muscle tissue, skeletal tissue, blood components, an extracellular matrix, a gland, an organ, and/or a system.

31. A method according to claim 17, wherein the inflammatory and/or degenerative process is an inflammatory process selected from the group consisting of bronchial asthma, allergic aveolitis, atopic dermatitis, auto-immune dermatitis, allergic chronic contact dermatitis, environmental chronic contact dermatitis, chronic laminitis, pemphigus, Bullous pemphigoid, equine reactive airway disease, chronic obstructive pulmonary disease, inflammatory airway disease, recurrent airway obstruction, summer pasture associated obstructive pulmonary disease, Crohn's disease, ulcerative colitis, immune mediated multiple sclerosis, environmental multiple sclerosis, autoimmune rheumatoid arthritis, periodontal disease, systemic lupus erythematosus, sarcoidosis, psoriasis, type I diabetes, ischemia-reperfusion injury, and combinations thereof.

32. A method according to claim 17, wherein the inflammatory and/or degenerative process is a degenerative process of lung tissue, skin tissue, bowel tissue, lamellar tissue, nerve tissue, connective tissue, vascular tissue, muscle tissue, skeletal tissue, blood components, an extracellular matrix, a gland, an organ, and/or a system.

33. A method according to claim 17, wherein the inflammatory and/or degenerative process is a degenerative process selected from the group consisting of Alzheimer's disease, atherosclerosis, arteriosclerosis, osteoarthritis, Huntington's chorea, Parkinson's disease, optic atrophy, retinitis pigmentosa, macular degeneration, muscular dystrophy, degenerative processes associated with aging, and combinations thereof.

34. A method according to claim 17, wherein the inflammatory and/or degenerative process is selected from the group consisting of chronic inflammatory disease, geriatric wasting, cancer cachexia, cachexia associated with chronic inflammation, sick feeling syndrome, and combinations thereof.

35. A composition for treating an inflammatory and/or degenerative process in a human or other animal, said composition comprising:
a curcuminoid;
a polymethoxylated flavone;
a catechin; and
a boswellic acid.

36. A composition according to claim 35, wherein said composition further comprises:
a Harapagophytum procumbens extract.

37. A composition according to claim 35, wherein said composition further comprises:
a Trypterigium wilfordii Hook extract.

38. A composition according to claim 35, wherein said composition further comprises:
a Glycyrrhiza glabra extract.

39. A composition according to claim 35, wherein said composition further comprises:
a Cinnamomum cassia extract.

40. A composition according to claim 35, wherein said composition further comprises:
a Magnolia obovata extract.

41. A composition according to claim 35, wherein said composition further comprises:
a Magnolia officianalis extract.

42. A composition according to claim 35, wherein said composition further comprises:
a Euonymus alatus extract.

43. A composition according to claim 35, wherein said composition further comprises:
two or more extracts selected from the group consisting of a Harapagophytum procumbens extract, a Trypterigium wilfordii Hook extract, a Glycyrrhiza glabra extract, a Cinnamomum cassia extract, a Magnolia obovata extract, a Magnolia officianalis extract, and a Euonymus alatus extract.

44. A composition according to claim 35, wherein said composition further comprises:
a Harapagophytum procumbens extract;
a Trypterigium wilfordii Hook extract;
a Glycyrrhiza glabra extract;
a Cinnamomum cassia extract;
a Magnolia obovata extract;
a Magnolia officianalis extract; and
a Euonymus alatus extract.

45. A composition according to claim 35, wherein said composition is in a pill form.

46. A composition according to claim 35, wherein said composition is in a powder or granular form.

47. A composition according to claim 35, wherein said composition is in a liquid form.

48. A composition according to claim 35, wherein said composition is in a food preparation form.

49. A composition according to claim 35, wherein said composition is in a dietary supplement form.

50. A method for treating an inflammatory and/or degenerative process in a subject, said method comprising:
administering to the subject a composition according to claim 35.

51. A method according to claim 50, wherein the composition further comprises one or more extracts selected from the group consisting of a Harapagophytum procumbens extract, a Trypterigium wilfordii Hook extract, a Glycyrrhiza glabra extract, a Cinnamomum cassia extract, a Magnolia obovata extract, a Magnolia officianalis extract, and a Euonymus alatus extract.

52. A method according to claim 50, wherein the composition further comprises two or more extracts selected from the group consisting of a Harapagophytum procumbens extract, a Trypterigium wilfordii Hook extract, a Glycyrrhiza glabra extract, a Cinnamomum cassia extract, a Magnolia obovata extract, a Magnolia officianalis extract, and a Euonymus alatus extract.

53. A method according to claim 50, wherein the composition further comprises a Harapagophytum procumbens extract, a Trypterigium wilfordii Hook extract, a Glycyrrhiza glabra extract, a Cinnamomum cassia extract, a Magnolia obovata extract, a Magnolia officianalis extract, and a Euonymus alatus extract.

54. A method according to claim 50, wherein the composition is administered to the subject in the form of a pill.

55. A method according to claim 50, wherein the composition is administered to the subject in the form of a powder or granules.

56. A method according to claim 50, wherein the composition is administered to the subject in the form of a liquid.

57. A method according to claim 50, wherein the composition is administered to the subject in the form of a food preparation.

58. A method according to claim 50, wherein the subject is a human.

59. A method according to claim 50, wherein the subject is a non-human animal.

60. A method according to claim 50, wherein the subject is selected from the group consisting of a horse, a dog, and a cat.

61. A method according to claim 50, wherein the subject is a zoo animal.

62. A method according to claim 50, wherein the inflammatory and/or degenerative process is selected from the group consisting of Alzheimer's disease, atherosclerosis or arteriosclerosis, degenerative joint diseases, Huntington's chorea, Parkinson's disease, optic atrophy, retinitis pigmentosa, macular degeneration, muscular dystrophy, asthma, dermatitis, laminitis, pemphigoid, pemphigus, reactive airway disease, inflammatory bowel disease, multiple sclerosis, rheumatoid arthritis, periodontal disease, systemic lupus erythematosus, sarcoidosis, psoriasis, type I diabetes, ischemia-reperfusion injury, and combinations thereof.

63. A method according to claim 50, wherein the inflammatory and/or degenerative process is an inflammatory and/or degenerative joint process.

64. A method according to claim 50, wherein the inflammatory and/or degenerative process is an inflammatory and/or degenerative joint process selected from the group consisting of osteoarthritis and rheumatoid arthritis.

65. A method according to claim 50, wherein the inflammatory and/or degenerative process is a result of infectious agents, is a result of physical insult, is a result of tumorogenesis and/or metastasis, is a result of chemical insult, is a result of oxidative stress, is immune mediated, and/or is a degenerative processes associated with aging.

66. A method according to claim 50, wherein the inflammatory and/or degenerative process is an inflammatory process of lung tissue, skin tissue, bowel tissue, lamellar tissue, nerve tissue, connective tissue, vascular tissue, muscle tissue, skeletal tissue, blood components, an extracellular matrix, a gland, an organ, and/or a system.

67. A method according to claim 50, wherein the inflammatory and/or degenerative process is an inflammatory process selected from the group consisting of bronchial asthma, allergic aveolitis, atopic dermatitis, auto-immune dermatitis, allergic chronic contact dermatitis, environmental chronic contact dermatitis, chronic laminitis, pemphigus, Bullous pemphigoid, equine reactive airway disease, chronic obstructive pulmonary disease, inflammatory airway disease, recurrent airway obstruction, summer pasture associated obstructive pulmonary disease, Crohn's disease, ulcerative colitis, immune mediated multiple sclerosis, environmental multiple sclerosis, autoimmune rheumatoid arthritis, periodontal disease, systemic lupus erythematosus, sarcoidosis, psoriasis, type I diabetes, ischemia-reperfusion injury, and combinations thereof.

68. A method according to claim 50, wherein the inflammatory and/or degenerative process is a degenerative process of lung tissue, skin tissue, bowel tissue, lamellar tissue, nerve tissue, connective tissue, vascular tissue, muscle tissue, skeletal tissue, blood components, an extracellular matrix, a gland, an organ, and/or a system.

69. A method according to claim 50, wherein the inflammatory and/or degenerative process is a degenerative process selected from the group consisting of Alzheimer's disease, atherosclerosis, arteriosclerosis, osteoarthritis, Huntington's chorea, Parkinson's disease, optic atrophy, retinitis pigmentosa, macular degeneration, muscular dystrophy, degenerative processes associated with aging, and combinations thereof.

70. A method according to claim 50, wherein the inflammatory and/or degenerative process is selected from the group consisting of chronic inflammatory disease, geriatric wasting, cancer cachexia, cachexia associated with chronic inflammation, sick feeling syndrome, and combinations thereof.
